(19) United States
(12) Patent Application Publication
(10) Pub. No.: US 2004/0139501 A1
(43) Pub. Date: Jul. 15, 2004

(54) Lis promoter for expression of transgenes in floral tissues

(75) Inventors: Randal Hauptmann, Oswego, IL (US); Alan Blowers, St. Charles, IL (US); Robert Eisenreich, North Aurora, IL (US)

Correspondence Address: WOOD, PHILLIPS, KATZ, CLARK & MORTIMER, 500 W. MADISON STREET, SUITE 3800, CHICAGO, IL, 60661, US

(73) Assignee: Ball Horticultural Company

(21) Appl. No.: 10/376,931

(22) Filed: Feb. 28, 2003

Related U.S. Application Data
(60) Provisional application No. 60/361,192, filed on Mar. 1, 2002.

(51) Int. Cl. A01H 1/00; C12N 15/82; C12Q 1/68; C07H 21/04; C12N 9/10; C12N 5/04

(52) U.S. Cl. 800/287; 435/6; 435/69.1; 435/320.1; 435/419; 536/23.2; 435/193

(57) ABSTRACT

The present invention relates to an isolated promoter derived from a S-linalool synthase gene that can be used to confer high levels of expression to at least one operably linked polynucleotide(s) or selected gene(s) in at least one flower of a plant.

### RELATED APPLICATION INFORMATION

This application claims priority to U.S. Serial No. 60/361,192 filed on Mar. 1, 2002, the disclosure of which is herein incorporated by reference.

### FIELD OF THE INVENTION

The present invention relates to plant genetic engineering. More specifically, the present invention relates to an isolated promoter derived from a S-linalool synthase gene that is capable of directing high levels of expression of at least one polynucleotide or selected gene operably linked to said promoter in at least one flower of a plant, transgenes preferentially expressed in at least one flower of a plant, and transformed plants containing said transgenes.

### BACKGROUND OF THE INVENTION

Expression of transgenes in plant tissues requires the presence of an operably linked promoter that is functional within the plant. The choice of a promoter sequence determines when and where within the plant the transgene(s) is expressed. A number of different types of promoters are known in the art such as constitutive, inducible, and tissue-specific promoters. Constitutive promoters are utilized when continuous expression of a transgene is desired throughout the cells of a plant. Constitutive promoters known in the art include, but are not limited to, rice actin 1 (Wang et al., Molecular and Cellular Biology, 12(8):3399-3406 (1992)), U.S. Pat. No. 5,641,876), Cauliflower Mosaic Virus (CaMV) 35S RNA (Odell et al., Nature, 313:810-812 (1985)), CaMV 19S RNA (Lawton et al., Plant Mol. Biol., 49:95-106 (1987)), nos (Ebert et al., Proc. Natl. Acad. Sci. USA, 84:5745-5749 (1987)), Adhl (Walker et al., Proc. Natl. Acad. Sci. USA, 84:6624-6628 (1987)) and the like. Inducible promoters are utilized when gene expression in response to a stimulus is desired. Inducible promoters known in the art include, but are not limited to, abscisic acid ABA and turgor-inducible promoters, the promoter of the auxin-binding protein gene (Schwob et al., Plant J., 4(3): 423-432 (1993)), the UDP glucose flavonoid glycosyl-transferase gene promoter (Ralston et al., Genet., 119(1):185-197 (1988)), and the like. Tissue-specific promoters are utilized when expression in specific tissues or organs is desired. Examples of tissue-specific promoters known in the art, include, but are not limited to, lectin (Vodkin et al., Cell, 34:1023 (1983), Lindstron et al., Developmental Genetics, 11:160 (1990)), pea small subunit RuBP carboxylase (Poulsen et al., Mol. Gen. Genet., 205(2):193-200 (1986), Cashmore et al., Gen. Eng. of Plants, Plenum Press, New York 29-38 (1983)), Ti plasmid mannopine synthase (Langridge et al., Proc. Natl. Acad. Sci. USA, 86:3219-3223 (1989)), petunia chalcone isomerase (Van Tunen et al., EMBO J, 7:1257 (1988)), and the like.

While a number of different types of promoters are known in the art, there is a need for the discovery of new promoters with beneficial expression characteristics, such as the ability of directing high-level expression of exogenous genes in transgenic plants.

### SUMMARY OF THE INVENTION

In one embodiment, the present invention relates to an isolated polynucleotide that encodes a promoter. The promoter of the present invention is capable of initiating transcription of at least one operably linked polynucleotide or selected gene in at least one flower of a plant. This isolated polynucleotide has a sequence selected from the group consisting of: SEQ ID NO:2 and a sequence that hybridizes to SEQ ID NO:2 under stringent conditions. The present invention also contemplates fragments and variants of the above-described sequences. The stringent conditions under which a sequence can hybridize to SEQ ID NO:2 can be low or high stringency conditions. Low stringency conditions comprise a wash at 42° C. in a solution of 2×SSC, 0.5% (w/v) SDS for 30 minutes and then repeating said wash. High stringency conditions comprise a wash at 65° C. in a solution of 2×SSC, 0.5% (w/v) SDS for 30 minutes and then repeating said wash.

In a second embodiment, the present invention relates to an isolated polynucleotide comprising a sequence that encodes a promoter that is preferentially active in initiating transcription of at least one operably linked polynucleotide(s) or selected gene(s) in at least one flower of a plant. This isolated polynucleotide has a sequence selected from the group consisting of: SEQ ID NO:2 and a sequence that hybridizes to SEQ ID NO:2 under stringent conditions. The present invention also contemplates fragments and variants of the above-described sequences. The stringent conditions under which a sequence can hybridize to SEQ ID NO:2 can be of low or high stringency. Low stringency conditions comprise a wash at 42° C. in a solution of 2×SSC, 0.5% (w/v) SDS for 30 minutes and then repeating said wash. High stringency conditions comprise a wash at 65° C. in a solution of 2×SSC, 0.5% (w/v) SDS for 30 minutes and then repeating said wash.

In yet a further embodiment, the present invention contemplates an expression cassette that comprises the above-described isolated promoter. The expression cassette of the present invention comprises the above-described promoter operably linked to a polynucleotide sequence. This promoter is capable of initiating transcription and expression of said polynucleotide sequence in at least one flower of a plant transformed with the expression cassette. The polynucleotide sequence that is operably linked to the promoter is inserted into the expression cassette in either the sense or antisense orientation.

In yet a further embodiment, the present invention contemplates an expression vector that comprises the above-described expression cassette.

In yet a further embodiment, the present invention relates to a plant or plant parts, stably transformed with the above-described expression cassette. Plant parts that can be transformed include, but are not limited to, cells, protoplasts, cell tissue cultures, callus, cell clumps, embryos, pollen, ovules, petals, styles, stamens, leaves, roots, root tips and anthers. The plant that can be transformed can be a monocotyledonous or a dicotyledonous plant. Examples of monocotyledonous plants include, but are not limited to: Amaryllidaceae (Allium, Narcissus); Graminae, alternatively Poaceae, (Avena, Horedum, Oryza, Panicum, Pennisetum, Poa, Saccharum, Secale, Sorghum, Triticum, Zea). Examples of dicotyledonous plants include, but are not limited to: Apocynaceae (Catharanthus); Asteraceae, alternatively Compositae (Aster, Calendula, Callistephus, Cichorium, Coreopsis, Dahlia, Dendranthema, Gazania, Gerbera, Helianthus, Helichrysum, Lactuca, Rudbeckia, Tagetes, Zinnia); Balsaminaceae (Impatiens); Begoniaceae (Begonia); Caryophyllaceae (Dianthus); Chenopodiaceae (Beta, Spinacia); Cucurbitaceae (Citrullus, Curcurbita, Cucumis); Cruciferae (Alyssum, Brassica, Erysimum, Matthiola, Raphanus); Gentinaceae (Eustoma); Geraniaceae (Pelargonium); Euphorbiaceae (Poinsettia); Labiatae (Salvia); Leguminosae (Glycine, Lathyrus, Medicago, Phaseolus, Pisum); Liliaceae (Lilium); Lobeliaceae (Lobelia); Malvaceae (Abelmoschus, Gossypium, Malva); Plumbaginaceae (Limonium); Polemoniaceae (Phlox); Primulaceae (Cyclamen); Ranunculaceae (Aconitum, Anemone, Aquilegia, Caltha, Delphinium, Ranunculus); Rosaceae (Rosa); Rubiaceae (Pentas); Scrophulariaceae (Angelonia, Antirrhinum, Torenia); Solanaceae (Capsicum, Lycopersicon, Nicotiana, Petunia, Solanum); Umbelliferae (Apium, Daucus, Pastinaca); Verbenaceae (Verbena, Lantana); Violaceae (Viola).

In yet a further embodiment, the present invention also contemplates seed produced by the plants described above that contain the above-described expression cassette.

In still yet a further embodiment, the present invention relates to an expression cassette comprising a chimeric promoter that is operably linked to a polynucleotide sequence. The chimeric promoter used in this expression cassette comprises (a) a first polynucleotide capable of initiating transcription of at least one operably linked polynucleotide(s) or a selected gene of interest(s) in at least one flower of a plant, wherein said polynucleotide has a sequence selected from the group consisting of: SEQ ID NO:2 and a sequence that hybridizes to SEQ ID NO:2 under stringent conditions and fragments and variants of these sequences; and (b) at least a second polynucleotide sequence, wherein said polynucleotide is capable of initiating transcription of a polynucleotide sequence or selected gene in a plant. Optionally, the second polynucleotide sequence making up the chimeric promoter has a sequence selected from the group consisting of: SEQ ID NO:2 and a sequence that hybridizes to SEQ ID NO:2 under stringent conditions. Fragments and variants of these sequences are also contemplated by the scope of the present invention. The polynucleotide that is operably linked to the chimeric promoter can be inserted into the expression cassette in either the sense or antisense orientation.

In yet a further embodiment, the present invention relates to an expression vector comprising an expression cassette comprising the expression cassette described above comprising the chimeric promoter.

In yet still a further embodiment, the present invention relates to a plant or plant parts, stably transformed with the expression cassette described above comprising the chimeric promoter. Plant parts that can be transformed include, but are not limited to, cells, protoplasts, cell tissue cultures, callus, cell clumps, embryos, pollen, ovules, petals, styles, stamens, leaves, roots, root tips and anthers. The plant that can be transformed can be a monocotyledonous or a dicotyledonous plant. Examples of monocotyledonous plants include, but are not limited to: Amaryllidaceae (Allium, Narcissus); Graminae, alternatively Poaceae, (Avena, Horedum, Oryza, Panicum, Pennisetum, Poa, Saccharum, Secale, Sorghum, Triticum, Zea). Examples of dicotyledonous plants include, but are not limited to: Apocynaceae (Catharanthus); Asteraceae, alternatively Compositae (Aster, Calendula, Callistephus, Cichorium, Coreopsis, Dahlia, Dendranthema, Gazania, Gerbera, Helianthus, Helichrysum, Lactuca, Rudbeckia, Tagetes, Zinnia); Balsaminaceae (Impatiens); Begoniaceae (Begonia); Caryophyllaceae (Dianthus); Chenopodiaceae (Beta, Spinacia); Cucurbitaceae (Citrullus, Curcurbita, Cucumis); Cruciferae (Alyssum, Brassica, Erysimum, Matthiola, Raphanus); Gentinaceae (Eustoma); Geraniaceae (Pelargonium); Euphorbiaceae (Poinsettia); Labiatae (Salvia); Leguminosae (Glycine, Lathyrus, Medicago, Phaseolus, Pisum); Liliaceae (Lilium); Lobeliaceae (Lobelia); Malvaceae (Abelmoschus, Gossypium, Malva); Plumbaginaceae (Limonium); Polemoniaceae (Phlox); Primulaceae (Cyclamen); Ranunculaceae (Aconitum, Anemone, Aquilegia, Caltha, Delphinium, Ranunculus); Rosaceae (Rosa); Rubiaceae (Pentas); Scrophulariaceae (Angelonia, Antirrhinum, Torenia); Solanaceae (Capsicum, Lycopersicon, Nicotiana, Petunia, Solanum); Umbelliferae (Apium, Daucus, Pastinaca); Verbenaceae (Verbena, Lantana); Violaceae (Viola).

In yet a further embodiment, the present invention also contemplates seed produced by the plants described above that contain the above-described expression cassette that comprises the chimeric promoter.

In yet still a further embodiment, the present invention relates to a transgenic plant cell stably transformed with a DNA molecule comprising a promoter capable of directing transcription in at least one flower of a plant. The promoter comprises a polynucleotide having a sequence selected from the group consisting of SEQ ID NO:2 and a sequence that hybridizes to SEQ ID NO:2 under stringent conditions. Fragments and variants of these sequences are also contemplated by the present invention. The DNA molecule used to transform the plant cell further comprises a selected coding region that is operable linked to the promoter. This selected coding region is either in the sense or antisense orientation. Additionally, the selected coding region can encode an insect resistance protein, a bacterial disease resistance protein, a fungal disease resistance protein, a viral disease resistance protein, an anthocyanin biosynthetic enzyme, a carotenoid biosynthetic enzyme, a floral scent biosynthetic protein, a screenable marker protein or a protein that promotes flower longevity. If the selected coding region encodes a screenable marker, said screenable marker can be selected from the group consisting of: beta-glucuronidase, beta-lactamase, beta-galactosidase, luciferase, aequorine and green fluorescent protein. If the selected coding region encodes an anthocyanin biosynthetic enzyme, said anthocyanin biosynthetic enzyme is capable of producing the compounds pelargonidin, cyanidin, delphinidin, peonidin, malvidin, and petunidin. If the selected coding region encodes a carotenoid biosynthetic enzyme, said carotenoid biosynthetic enzyme is capable of producing the compounds: phytoene, phytofluene, ζ-carotene, neurosporene, lycopene, γ-carotene, β-carotene, α-cryptoxanthin, β-cryptoxanthin, canthaxanthin, capsanthin, capsorubin, zeaxanthin, violaxanthin, neoxanthin, antheraxanthin, lutein, astaxanthin, adonirubin and adonixanthin.

The stringent conditions under which a sequence can hybridize to SEQ ID NO:2 can be low or high stringency conditions. Low stringency conditions comprise a wash at 42° C. in a solution of 2×SSC, 0.5% (w/v) SDS for 30 minutes and then repeating said wash. High stringency conditions comprise a wash at 65° C. in a solution of 2×SSC, 0.5% (w/v) SDS for 30 minutes and then repeating said wash.

In yet another embodiment, the present invention relates to a method for selectively expressing a first polynucleotide sequence in at least one flower of a transgenic plant. The method involves the step of: transforming a plant or plant cell with an expression vector comprising an expression cassette, said expression cassette comprising a promoter and a first polynucleotide sequence operably linked to said promoter, wherein the promoter is capable initiating transcription and directing expression of said first polynucleotide sequence in at least one flower of a plant and comprises a polynucleotide having a sequence having a sequence selected from the group consisting of SEQ ID NO:2 and a sequence that hybridizes to SEQ ID NO:2 under stringent conditions. Fragments and variants of these sequences are also contemplated by the scope of the present invention. The first polynucleotide is inserted in the expression cassette in either the sense or antisense orientation.

The stringent conditions under which a sequence can hybridize to SEQ ID NO:2 can be low or high stringency conditions. Low stringency conditions comprise a wash at 42° C. in a solution of 2×SSC, 0.5% (w/v) SDS for 30 minutes and then repeating said wash. High stringency conditions comprise a wash at 65° C. in a solution of 2×SSC, 0.5% (w/v) SDS for 30 minutes and then repeating said wash.

The first polynucleotide sequence inserted in the expression cassette can encode an insect resistance protein, a bacterial disease resistance protein, a fungal disease resistance protein, a viral disease resistance protein, an anthocyanin biosynthetic enzyme, a carotenoid biosynthetic enzmye, a floral scent biosynthetic protein, a screenable marker protein or a protein that promotes flower longevity. If the first polynucleotide sequence encodes a screenable marker protein, then said screenable marker protein can be selected from the group consisting of: beta-glucuronidase, beta-lactamase, beta-galactosidase, luciferase, aequorine and green fluorescent protein. If the first polynucleotide sequence encodes an anthocyanin biosynthetic enzyme, then said anthocyanin enzyme is capable of producing the compounds: pelargonidin, cyanidin, delphinidin, peonidin, malvidin, and petunidin. If the first polynucleotide sequence encodes a carotenoid biosynthetic enzyme, then said biosynthetic enzyme is capable of producing the compunds: phytoene, phytofluene, ζ-carotene, neurosporene, lycopene, γ-carotene, β-carotene, α-cryptoxanthin, β-cryptoxanthin, canthaxanthin, capsanthin, capsorubin, zeaxanthin, violaxanthin, neoxanthin, antheraxanthin, lutein, astaxanthin, adonirubin and adonixanthin.

The plant or plant cell that is transformed can be from a monocotyledonous plant, including, but not limited to: Amaryllidaceae (Allium, Narcissus); Graminae, alternatively Poaceae, (Avena, Horedum, Oryza, Panicum, Pennisetum, Poa, Saccharum, Secale, Sorghum, Triticum, Zea). Alternatively, the plant or plant cell that is transformed can be from a dicotyledonous plant, including, but not limited to: Apocynaceae (Catharanthus); Asteraceae, alternatively Compositae (Aster, Calendula, Callistephus, Cichorium, Coreopsis, Dahlia, Dendranthema, Gazania, Gerbera, Helianthus, Helichrysum, Lactuca, Rudbeckia, Tagetes, Zinnia); Balsaminaceae (Impatiens); Begoniaceae (Begonia); Caryophyllaceae (Dianthus); Chenopodiaceae (Beta, Spinacia); Cucurbitaceae (Citrullus, Curcurbita, Cucumis); Cruciferae (Alyssum, Brassica, Erysimum, Matthiola, Raphanus); Gentinaceae (Eustoma); Geraniaceae (Pelargonium); Euphorbiaceae (Poinsettia); Labiatae (Salvia); Leguminosae (Glycine, Lathyrus, Medicago, Phaseolus, Pisum); Liliaceae (Lilium); Lobeliaceae (Lobelia); Malvaceae (Abelmoschus, Gossypium, Malva); Plumbaginaceae (Limonium); Polemoniaceae (Phlox); Primulaceae (Cyclamen); Ranunculaceae (Aconitum, Anemone, Aquilegia, Caltha, Delphinium, Ranunculus); Rosaceae (Rosa); Rubiaceae (Pentas); Scrophulariaceae (Angelonia, Antirrhinum, Torenia); Solanaceae (Capsicum, Lycopersicon, Nicotiana, Petunia, Solanum); Umbelliferae (Apium, Daucus, Pastinaca); Verbenaceae (Verbena, Lantana); Violaceae (Viola).

In yet a further embodiment, the present invention relates to a method of expressing a selected protein in at least one flower of a transgenic plant. The first step of the method involves obtaining an expression vector comprising a selected coding region operably linked to a promoter capable of initiating transcription in a flower of a plant. The promoter comprises a polynucleotide having a sequence selected from the group consisting of SEQ ID NO:2 and a sequence that hybridizes to SEQ ID NO:2 under stringent conditions. Fragments and variants of these sequences are also contemplated by the scope of the present invention. The second step involves transforming a recipient plant cell with said vector. The third step involves regenerating a transgenic plant expressing said selected protein from said recipient plant cell.

The selected coding region employed in the expression cassette can be in either the sense or antisense orientation. The stringent conditions under which a sequence can hybridize to SEQ ID NO:2 can be low or high stringency conditions. Low stringency conditions comprise a wash at 42° C. in a solution of 2×SSC, 0.5% (w/v) SDS for 30 minutes and then repeating said wash. High stringency conditions comprise a wash at 65° C. in a solution of 2×SSC, 0.5% (w/v) SDS for 30 minutes and then repeating said wash.

The transformation of the plant cell can be conducted using techniques known in the art, including, but not limited to, microprojectile bombardment, polyethylene glycol-mediated transformation of protoplasts, electroporation and Agrobacterium-mediated transformation. The recipient plant cell being transformed can be from either a monocotyledonous or a dicotyledonous plant. Examples of monocotyledonous plants include, but are not limited to: Amaryllidaceae (Allium, Narcissus); Graminae, alternatively Poaceae, (Avena, Horedum, Oryza, Panicum, Pennisetum, Poa, Saccharum, Secale, Sorghum, Triticum, Zea). Examples of dicotyledonous plants include, but are not limited to: Apocynaceae (Catharanthus); Asteraceae, alternatively Compositae (Aster, Calendula, Callistephus, Cichorium, Coreopsis, Dahlia, Dendranthema, Gazania, Gerbera, Helianthus, Helichrysum, Lactuca, Rudbeckia, Tagetes, Zinnia); Balsaminaceae (Impatiens); Begoniaceae (Begonia); Caryophyllaceae (Dianthus); Chenopodiaceae (Beta, Spinacia); Cucurbitaceae (Citrullus, Curcurbita, Cucumis); Cruciferae (Alyssum, Brassica, Erysimum, Matthiola, Raphanus); Gentinaceae (Eustoma); Geraniaceae (Pelargonium); Euphorbiaceae (Poinsettia); Labiatae (Salvia); Leguminosae (Glycine, Lathyrus, Medicago, Phaseolus, Pisum); Liliaceae (Lilium); Lobeliaceae (Lobelia); Malvaceae (Abelmoschus, Gossypium, Malva); Plumbaginaceae (Limonium); Polemoniaceae (Phlox); Primulaceae (Cyclamen); Ranunculaceae (Aconitum, Anemone, Aquilegia, Caltha, Delphinium, Ranunculus); Rosaceae (Rosa); Rubiaceae (Pentas); Scrophulariaceae (Angelonia, Antirrhinum, Torenia); Solanaceae (Capsicum, Lycopersicon, Nicotiana, Petunia, Solanum); Umbelliferae (Apium, Daucus, Pastinaca); Verbenaceae (Verbena, Lantana); Violaceae (Viola).

### BRIEF DESCRIPTION OF THE FIGURES

FIG. 1A shows the polynucleotide sequence of the LIS1 promoter with the oligonucleotide primers (BHX30 and BHX36). The polynucleotide sequence shown in FIG. 1A is 1048 base pairs in length and contains a Hind III site at nucleotides 3-8 and a Sma I site at nucleotides 1040-1045.

FIG. 1B shows the nucleotide sequence of the oligonucleotide primer BHX30. This primer constitutes nucleotides 1-29 of the polynucleotide sequence shown in FIG. 1A.

FIG. 1C shows the nucleotide sequence of the oligonucleotide primer BHX36. This primer is the reverse complement of nucleotides 1018-1048 of the polynucleotide sequence shown in FIG. 1A.

FIG. 2 shows photographs of differences in GUS expression between transgenic petunia lines transformed with plasmids containing the LIS1 promoter and plasmids containing the 35S RNA constitutive promoter.

FIG. 3 shows an RNA gel blot analysis of petunia flower sections from plants transformed with pBHX112 containing LIS1::crtB::nos.

### DETAILED DESCRIPTION OF THE INVENTION

Introduction

The present invention relates to the use of a polynucleotide sequence derived from a S-linalool synthase gene as a promoter to initiate transcription in specific tissues, such as in at least one flower of a plant. The polynucleotide sequence of the present invention can be used to express a protein of interest in at least one flower of a plant.

Definitions

The headings provided herein are not limitations of the various aspects or embodiments of the invention that can be had by reference to the specification as a whole. Accordingly, the terms defined immediately below are more fully defined by reference to the specification as a whole.

As used herein, the phrases, “exogenous coding region” or “selected coding region” refers to a coding region of a polynucleotide(s) or selected gene(s) that is introduced or re-introduced into an organism. For example, a coding region (from a polynucleotide(s) or selected gene(s)) that encodes for the carotenoid lutein is considered an exogenous coding region or selected coding region if it is introduced or re-introduced into an organism, such as a plant, such as marigold.

As used herein, the term “expression” refers to the combination of intracellular processes, including transcription and translation undergone by a coding DNA molecule such as a structural gene or a polynucleotide to produce a polypeptide.

As used herein, the term “expression cassette” refers to a chimeric DNA molecule that is designed for introduction into a host genome by genetic transformation. Preferred expression cassettes of the present invention comprise all the genetic elements necessary to direct the expression of a selected gene or polynucleotide sequence. The expression cassette(s) of the present invention include a LIS1 promoter or a LIS1 chimeric promoter.

As used herein, the term “expression vector” refers to a DNA-based vector comprising at least one expression cassette.

As used herein, the term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

As used herein, the term “host” or “hosts” refers to bacteria, entire plants, plantlets, or plant parts such as plant cells, protoplasts, calli, roots, tubers, propagules, seeds, seedlings, pollen and plant tissues.

As used herein, the term “isolated” refers to material, such as a polynucleotide or protein that is (a) substantially or essentially free from components that normally accompany or interact with the material as found in its naturally occurring environment; or (b) if the material is in its natural environment, the material has been altered by deliberate human intervention to a composition and/or placed at a locus in a cell other than the locus native to the material.

As used herein, the term “marker genes” refers to genes that impart a distinct phenotype to cells expressing the marker gene and allow transformed cells to be distinguished from cells that do not have the marker gene. Such genes can encode a screenable marker that one can identify through observation or testing (i.e. such as by screening, such as the green fluorescent protein).

As used herein, the term “tissue-preferred” refers to polynucleotide or selected gene expression that has the highest level in any group of cells that perform a particular function. Techniques for determining the highest level of polynucleotide(s) or gene(s) expression in a group of cells are known in the art and include, but are not limited to, histochemical and fluorometric assays.

As used herein, the term “flower-preferred” refers to favored expression of at least one polynucleotide or selected gene in at least one flower of a plant.

As used herein, the term “petal-preferred” refers to polynucleotide or selected gene expression that has the highest level in floral petal tissue. Techniques for determining the highest level of polynucleotide or selected gene expression in a group of cells are known in the art and include, but are not limited to, histochemical and fluorometric assays.

As used herein, the term “plant part(s)” or “part(s) of a plant” refers to cells, protoplasts, cell tissue cultures, callus (calli), cell clumps, embryos, pollen, ovules, petals, styles, stamens, leaves, roots, root tips and anthers.

As used herein, the term “polynucleotide” refers to a polymeric form of nucleotides of any length, either ribonucleotides or deoxybribonucleotides. This term includes double- and single-stranded DNA, as well as, double- and single-stranded RNA. It also includes modifications, such as methylation or capping and unmodified forms of the polynucleotide.

As used herein, the term “promoter” refers to a recognition site on a DNA sequence or group of DNA sequences that provide an expression control element for a polynucleotide or selected gene and to which RNA polymerase specifically binds and initiates transcription (RNA synthesis) of that gene. Promoters typically consist of several regulatory elements involved in initiation, regulation, and efficiency of transcription that may be hundreds or even thousands of nucleotides proximal to the site of transcription initiation. Such regulatory elements include, but are not limited to, a TATA box, enhancers, upstream activation sequences, etc.

As used herein, the term “selected gene” refers to a gene that is to be expressed in a transgenic plant, plant cell or plant part. A selected gene can be native or foreign to a host genome. When the selected gene is present in the host genome, it includes one or more regulatory or functional elements that differ from the native copy(ies) of the gene.

As used herein, the term “transformed cell” refers to a cell, the DNA complement of which, has been altered by the introduction of an exogenous DNA molecule (i.e. exogenous coding region) into that cell. The “exogenous DNA molecule” includes (1) sequence(s) not originally present in the cell; and (2) sequences that are native to the cell being transformed and are being re-introduced to said cell.

As used herein, the term “transgene” refers to a segment of DNA that has been incorporated into a host genome or is capable of autonomous replication in a host cell and is capable of causing the expression of one or more cellular products.

As used herein, the term “transgenic plant” refers to a plant or progeny from a plant of any subsequent generation derived therefrom, where the DNA of the plant or progeny therefrom contains an introduced exogenous DNA molecule not originally present in a non-transgenic plant of the same strain. The transgenic plant can also contain sequences, that are native to the plant being transformed, but where the “exogenous” gene has been altered in order to change the level or pattern of expression of the gene.

As used herein, the term “transit peptide” refers to a polypeptide sequence that is capable of directing a polypeptide to a particular organelle or other location within a cell.

As used herein, the term “vector” refers to a DNA molecule capable of replication in a host cell and/or to which another DNA segment can be operatively linked in order to bring about replication of the attached segment. A plasmid is an example of a vector.

Sequence Listings

The present application also contains 5 polynucleotide and/or amino acid sequence. For the polynucleotide sequences, the base pairs are represented by the following base codes:

| Symbol | Meaning |
|---|---|
| A | adenine |
| C | cytosine |
| G | guanine |
| T | thymine |
| U | uracil |
| M | A or C |
| R | A or G |
| W | A or T/U |
| S | C or G |
| Y | C or T/U |
| K | G or T/U |
| V | A or C or G; not T/U |
| H | A or C or T/U; not G |
| D | A or G or T/U; not C |
| B | C or G or T/U; not A |
| N | (A or C or G or T/U) |

The amino acids shown in the application are in the L-form and are represented by the following amino acid-three letter abbreviations:

| Abbreviation | Amino acid name |
|---|---|
| Ala | L-Alanine |
| Arg | L-Arginine |
| Asn | L-Asparagine |
| Asp | L-Aspartic Acid |
| Asx | L-Aspartic Acid or Asparagine |
| Cys | L-Cysteine |
| Glu | L-Glutamic Acid |
| Gln | L-Glutamine |
| Glx | L-Glutamine or Glutamic Acid |
| Gly | L-Glycine |
| His | L-Histidine |
| Ile | L-Isoleucine |
| Leu | L-Leucine |
| Lys | L-Lysine |
| Met | L-Methionine |
| Phe | L-Phenylalanine |
| Pro | L-Proline |
| Ser | L-Serine |
| Thr | L-Threonine |
| Trp | L-Tryptophan |
| Tyr | L-Tyrosine |
| Val | L-Valine |
| Xaa | L-Unknown or other |

### DESCRIPTION OF THE PREFERRED EMBODIMENTS

In one embodiment, the present invention relates to the use an isolated polynucleotide sequence derived from a S-linalool synthase (LIS) gene that encodes a promoter. The polynucleotide sequence described herein is capable of directing the expression of potentially any operably linked polynucleotide(s) or selected gene(s) of interest in at least one flower of a plant. Such expression has been found to be not only flower-preferred, but more specifically, to be petal-preferred.

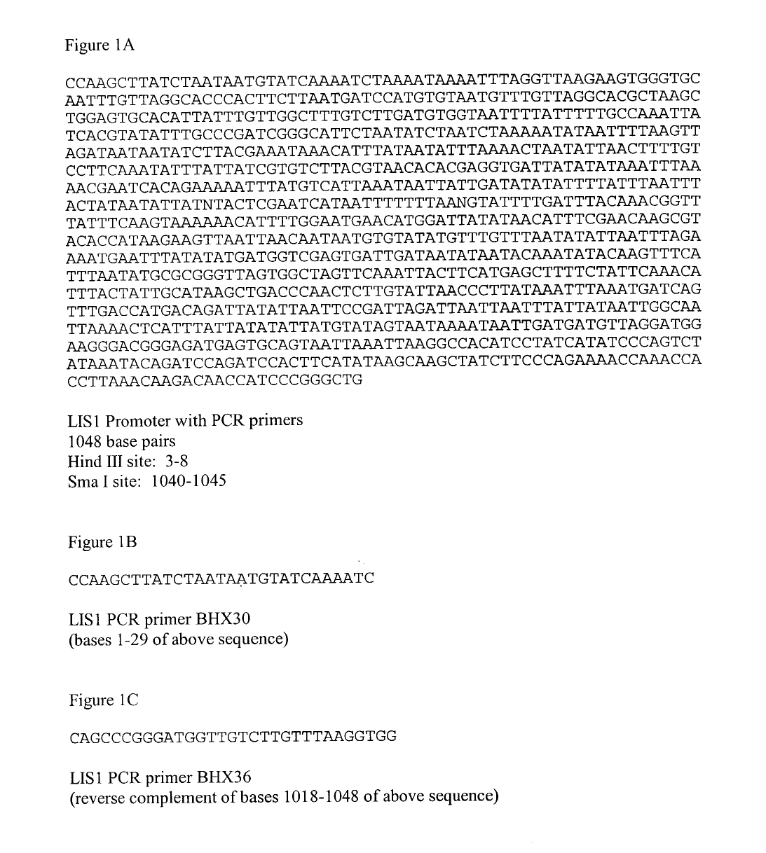

The polynucleotide sequence that encodes the promoter of the present invention, is shown in SEQ ID NO:2 and nucleotides 7-1033 of FIG. 1 (hereinafter “LIS1 promoter”). This polynucleotide sequence was derived from the S-linalool synthase gene from Clarkia brewerii (SEQ ID NO: 1). The entire polynucleotide and amino acid sequence for the S-linalool synthase gene from Clarkia brewerii is described in Genbank Accession AF067601 (SEQ ID NO:1) and Cseke, L., et al., Mol. Biol. Evol., 15:1491-1498 (1998). The S-linalool synthase gene encodes for S-linalool synthase, a floral scent biosynthetic enzyme that catalyzes the production of S-linalool, a volatile monoterpenoid (Dudareva et al, The Plant Cell, 8:1137-1148 (1996)).

Another floral compound, benzoic acid carboxylmethyl transferase, is the final enzyme in the biosynthesis of methyl benzoate, and is known to be the most abundant scent compound in snapdragon flowers. In snapdragon, the majority of BAMT gene activity was found in the upper and lower lobes of the corolla (Dudareva et al., The Plant Cell, 12:949-961 (2000)). An example demonstrates that the promoter from the BAMT gene did not direct foreign gene expression in transgenic petunia petal tissue. Therefore, this promoter cannot be assumed to be a petal-preferred promoter in other species.

The promoter of the present invention can be used to isolate other promoters from other organisms using routine techniques known in the art based on their sequence homology to the sequence of the promoter of the present invention. In these techniques, all or part of a the promoter of the present invention is used as a probe that selectively hybridizes to other sequences that are unique and are preferably at least about 10 nucleotides in length, and most preferably at least about 20 nucleotides in length. These probes can be used to amplify corresponding promoter from a chosen organism using the polymerase chain reaction (“PCR”). This technique can be used to isolate additional promoters from a desired organism or as a diagnostic assay to determine the presence of the promoter in an organism. Examples include hybridization screening of plated DNA libraries (either plaques or colonies; see e.g. Innis et al., PCR Protocols, A Guide to Methods and Applications, eds., Academic Press (1990)).

The present invention also encompasses sequences that correspond to the promoter of the present invention and hybridize to the promoter of the present invention and are at least 50% homologous, 70% homologous, 85% homologous 90% homologous, 95% homologous, and even 99% homologous with the disclosed sequence. That is, the sequence similarity between probe and target may range, sharing at least about 50%, about 70%, about 85%, about 90%, about 95% and even about 95% sequence similarity.

Methods of aligning sequences for comparison are well-known in the art. Gene comparisons can be determined by conducting BLAST (Basic Local Alignment Search Tool; Altschul, S. F., et al., J. Mol. Biol. 215:403-410 (1993); see also www.ncbi.nlm.nih.gov/BLAST/) searches under default parameters for identity to sequences contained in the BLAST “GENEMBL” database. A sequence can be analyzed for identity to all publicly available DNA sequences contained in the GENEMBL database using the BLASTN algorithm under the default parameters. Identity to the sequence of the present invention would mean a polynucleotide sequence having at least 50% sequence identity, more preferably at least 70% sequence identity, more preferably at least 75% sequence identity, more preferably at least 80% identity, more preferably at least 85% sequence identity, more preferably at least 90% sequence identity, more preferably at least 95% sequence identity and most preferably at least 99% sequence identity wherein the percent sequence identity is based on the entire promoter.

The identification of polynucleotide sequences that hybridize to the polynucleotide sequence of the present invention (SEQ ID NO:2) can be made using routine techniques in the art, such as through the use of stringent conditions. As used herein, the terms, “stringent conditions” or “stringent hybridization conditions” includes reference to conditions under which a probe will hybridize to its target sequence, to a detectably greater degree than other sequences (e.g., at least 2-fold over background). Stringent conditions are target sequence dependent and differ depending on the structure of a polynucleotide. By controlling the stringency of the hybridization and/or washing conditions, target sequences can be identified which are 100% complementary to a probe (this type of probing is known as “homologous probing”). Alternatively, stringency conditions can be adjusted to allow some mismatching in sequences so that lower degrees of similarity are detected (this type of probing is known as “heterologous probing”). Generally, probes of this type are in a range of about 100 nucleotides in length to about 250 nucleotides in length.

An extensive guide to the hybridization of nucleic acids is found in Tijssen, Laboratory Techniques in Biochemistry and Molecular Biology-Hybridization with Nucleic Acid Probes, Part I, Chapter 2, “Overview of principles of hybridization and the strategy of nucleic acid probe assays”, Elsevier, N.Y. (1993); and Current Protocols in Molecular Biology, Chapter 2, Ausubel, et al., Eds., Greene Publishing and Wiley-Interscience, New York (1995). See also Sambrook et al. Molecular Cloning: A Laboratory Manual (2^{nd} ed. Cold Spring Harbor Laboratory, Cold Spring Harbor, N.Y. (1989)).

Specificity is typically the function of post-hybridization washes, the critical factors being the ionic strength and temperature of the final wash solution. Generally, stringent wash temperature conditions are selected to be about 5° C. to about 2° C. lower than the melting point (Tₘ) for the specific sequence at a defined ionic strength and pH. The melting point, or denaturation, of DNA occurs over a narrow temperature range and represents the disruption of the double helix into its complementary single strands. The process is described by the temperature of the midpoint of transition, Tₘ, which is also called the melting temperature. Formulas for determining the melting temperatures are known in the art.

Preferred hybridization conditions for the promoter of the invention include hybridization at 42° C. in 50% (w/v) formamide, 6× standard saline citrate (“SSC”), 0.5% (w/v) sodium dodecyl sulfate (“SDS”), 100 μg/ml salmon sperm DNA. Exemplary low stringency washing conditions include hybridization at 42° C. in a solution of 2× SSC, 0.5% (w/v) SDS for 30 minutes and repeating. Exemplary moderate stringency conditions include a wash in 2× SSC, 0.5% (w/v) SDS at 50° C. for 30 minutes and repeating. Exemplary high stringency conditions include a wash in 2×SSC, 0.5% (w/v) SDS, at 65° C. for 30 minutes and repeating. Sequences that correspond to the promoter of the present invention may be obtained using all the above conditions.

The present invention also contemplates fragments and variants derived from the promoter of the present invention (namely, SEQ ID NO:2). As used herein, “fragment(s)” refers to a portion of a polynucleotide sequence. The fragment(s) of the present invention comprise a portion of SEQ ID NO:2. These fragments can retain biological activity and hence encompass fragments that are capable of directing expression of an operably linked polynucleotide(s) or selected gene(s) in at least one flower of a plant. Polynucleotide fragments of the promoter of the present invention comprise at least 20, 50, 75, 100, 150, 200, 250, 300, 350, 400, 450, 500, 550, 600, 650, 700, 750, 800, 850, 900, 950, 1000, 1010 nucleotides, or up to the number of nucleotides present in the full-length LIS1 promoter disclosed herein. The polynucleotide fragments will usually comprise the TATA recognition sequence (also known as a TATA box) of the particular promoter sequence. Such fragments can be obtained using restriction enzymes to cleave the polynucleotide sequence of the promoter disclosed herein; by synthesizing a polynucleotide sequence from the naturally occurring promoter DNA sequence; or can be obtained through the use of PCR technology. See particularly, Mullis et al. Methods Enzymol. 155:335-350 (1987), and Erlich, edl. (1989) PCR Technology (Stockton Press, N.Y. (1989)).

As discussed briefly above, the present invention also contemplates variants of the promoter of the present invention (SEQ ID NO:2). As used herein, the term “variant(s)” refers to a substantially similar sequence. Naturally-occurring variants can be identified using routine techniques known in the art, such as polymerase chain reaction. Variant polynucleotide sequences also include synthetically derived polynucleotide sequences, such as those produced by site-directed mutagenesis, which is described in more detail below. Biologically active variants are also encompassed by the scope of the present invention.

Variants of the promoter of the present invention can be produced by inserting, deleting or mutating SEQ ID NO:2 using routine techniques known in the art. For example, as discussed briefly above, such variants include sequences resulting from site-directed mutagenesis of SEQ ID NO:2. Techniques for carrying out site-directed mutagenesis are described in Mikaelian et al., Nucl. Acids Res., 20:376 (1992), Zhou et al., Nucl. Acids Res., 19:6052 (1991). Additionally, the present invention encompasses variants of the 5′ portion of a promoter up to the TATA box near the transcription start site that can be deleted without abolishing promoter activity, as described by Zhu et al., The Plant Cell 7: 1681-89 (1995). Such variants should retain the ability to direct expression in at least one flower of a plant.

The polynucleotide sequences of the present invention can be used in directing the expression in at least one flower of a plant of a selected coding region from an operably linked polynucleotide(s) and/or selected gene(s) that encodes a specific protein or polypeptide product or RNA molecule. The choice of a particular selected coding region used in conjunction with the LIS1 promoter for transformation of recipient plant cells will depend on the purpose of the transformation. One of the main purposes of transformation of plants is to add commercially desirable, agronomically or horticulturally important traits to a plant. Such traits include, but are not limited to, insect resistance or tolerance, disease resistance or tolerance (viral, bacterial, fungal), color (such as (1) anthocyanins (such as through the production or increased expression of anthocyanin biosynthetic enzymes that produce anthocyanin compounds, such as, but not limited to: pelargonidin, cyanidin, delphinidin, peonidin, malvidin, petunidin and the like (Polynucleotide and gene sequences that encode anthocyanin biosynthetic enzymes that produce the above-described compounds are known in the art and described in Holton et al., The Plant Cell, 7:1071-1083 (1995)) and/or (2) carotenoids (such as through the production or increased expression of carotenoid biosynthetic enzymes that produce compounds such as, but not limited to, phytoene, phytofluene, ζ-carotene, neurosporene, lycopene, γ-carotene, β-carotene, α-cryptoxanthin, β-cryptoxanthin, canthaxanthin, capsanthin, capsorubin, zeaxanthin, violaxanthin, neoxanthin, antheraxanthin, lutein, astaxanthin, adonirubin, adionixanthin and the like (Polynucleotides and gene sequences that encode for carotenoid biosynthetic enzymes that produce the above-described carotenoid compounds are known in the art and are described in WO 00/32788 and U.S. Pat. Nos. 5,684,238, 5,530,188, 5,429,939 and 5,618,988, Cunningham and Gantt, Breeding for Ornamentals: Classical and Molecular Approaches, ed. A. Vainstein (Kluwer Academic Publishers, Dordrecht (2002)), Chamovitz, et al., FEBS Lett., 296(3):305-310 (1992), Chappell, J., Ann. Rev. Plant Physiol. Plant Mol. Biol., 46:521-547 (1995), Cunningham et al., FEBS Lett., 328(1-2):130-138 (1993), Hugueney, et al., Eur. J. Biochem., 209(1):399-407 (1992), Hundle et al., FEBS Lett., 315(3):329-334 (1993), Kajiwara et al., Plant Mol. Biol., 29(2):343-352 (1995), Kuntz, et al., Plant J., 2(1):25-34 (1992), Linden et al., Plant Mol. Biol., 24(2):369-79 (1994), Lotan et al., FEBS Lett., 364(2):125-128 (1995), Math et al., Proc. Natl. Acad. Sci. USA, 89(15):6761-6764 (1992), Misawa et al., J. Bacteriol., 172(12):6704-6712 (1990), Misawa et al., J. Biochem. (Tokyo), 116(5):980-985 (1994), Sandmann, G., FEMS Microbiol. Lett., 69(3):253-257 (1992), Sandmann et al., FEMS Microbiol. Lett., 59(1-2):77-82 (1990) and Kajiwara et al., Biochem. J, 324:421-426 (1997))), floral scent, flower longevity and the like. The selected coding region can be used with the promoter of the present invention in the sense or antisense orientation.

In yet another embodiment, the present invention relates to a chimeric promoter that can be used to direct the expression in at least one flower of a plant of a selected coding region from an operably polynucleotide(s) and/or selected gene(s) that encodes a specific protein or polypeptide product or RNA molecule. More specifically, the polynucleotide of the present invention or a fragment or variant thereof can be operably linked to another promoter using routine techniques known in the art to form a chimeric promoter (hereinafter referred to as “LIS1 chimeric promoter”). For example, the promoter of the present invention can be operably linked to specific regions of the CaMV35S promoter to direct high levels of expression of at least one polynucleotide or selected gene operably linked to said chimeric promoter in at least one flower of a plant. The promoter used to make the LIS1 chimeric promoter along with the LIS1 promoter of the present invention does not only have to direct expression of a polynucleotide sequence or selected gene in a flower of a plant. In fact, this promoter does not have to be only a tissue-specific promoter. Optionally, said promoter can be a constitutive promoter or an inducible promoter that is well known in the art.

The choice of a particular selected coding region used in conjunction with the LIS1 chimeric promoter for transformation will depend on the purpose of the transformation. One of the main purposes of transformation of plants is to add commercially desirable, agronomically or horticulturally important traits to a plant. Such traits include, but are not limited to, insect resistance or tolerance, disease resistance or tolerance (viral, bacterial, fungal), color (such as (1) anthocyanins (such as through the production or increased expression of anthocyanin biosynthetic enzymes that produce anthocyanin compounds, such as, but not limited to: pelargonidin, cyanidin, delphinidin, peonidin, malvidin, petunidin and the like) and/or (2) carotenoids (such as through the production or increased expression of carotenoid biosynthetic enzymes that produce carotenoid compounds such as, but not limited to, phytoene, phytofluene, ζ-carotene, neurosporene, lycopene, γ-carotene, β-carotene, α-cryptoxanthin, β-cryptoxanthin, canthaxanthin, capsanthin, capsorubin, zeaxanthin, violaxanthin, neoxanthin, antheraxanthin, lutein, astaxanthin, adonirubin, adionixanthin and the like)), floral scent, flower longevity and the like. The particular selected coding region can be used with the LIS1 chimeric promoter in the sense or antisense orientation.

In another embodiment, the present invention contemplates the transformation of a recipient cell with more than one transformation construct. Two or more transgenes can be created in a single transformation event using either distinct selected-protein encoding vectors, or using a single vector incorporating two or more polynucleotide or selected gene sequences. Any two or more transgenes of any description, such as those conferring, for example, insect or disease (viral, bacterial, fungal) resistance, modifications (reduction or enhancement) to color or floral scent or flower longevity may be employed as desired.

In yet another embodiment, the present invention contemplates the co-transformation of plants or plant cells with two (2) or more vectors. Co-transformation may be achieved using a vector containing the marker and one or more polynucleotide(s) or selected gene(s) of interest. Alternatively, different vectors (such as, but not limited to plasmids) can contain different polynucleotides or selected genes of interest, and the plasmids can be concurrently delivered to the recipient host cells. According to this method, it is assumed that a certain percentage of cells in which the marker has been introduced, also have received the other polynucleotide or selected gene(s) of interest. Thereupon, not all cells selected by means of the marker, will express the other proteins of interest that had been presented to the cells concurrently.

Any vector suitable for plant transformation can be used in the present invention. Such vectors include, but are not limited to, plasmids, cosmids, yeast artificial chromosomes (“YACs”), bacterial artificial chromosomes (“BACs”) or any other suitable cloning system. It is contemplated that utilization of cloning systems with large insert capacities will allow introduction of large DNA sequences comprising more than one selected gene. Introduction of such sequences may be facilitated by use of BACs, YACs, or plant artificial chromosomes.

Expression cassettes isolated from the previously described vectors can be used in transformation. DNA molecules used for transforming plant cells will, generally comprise the cDNA or one or more selected genes or polynucleotides that are to be introduced into and expressed in recipient host cells. The DNA molecules can further include, in addition to a LIS1 promoter or LIS1 chimeric promoter, structures such as enhancers, polylinkers, introns, terminators or other regulatory elements or genes that influence gene expression. The DNA molecule used for transforming plant cells can be inserted into the expression cassette in the sense or antisense orientation and will often encode a protein that will be expressed in the resultant recombinant cells resulting in a screenable or selectable trait and/or which will impart an improved phenotype to the resulting transgenic plant. However, this may not always be the case, and the present invention also encompasses transgenic plants and plant parts incorporating non-expressed transgenes or non-coding RNA's (such as antisense RNA molecules, polynucleotides that encode a ribozyme or polynucleotides that are capable of promoting RNase P-mediated cleavage of target RNA molecules).

As discussed above, enhancer sequences can be included in transformation constructs containing the LIS1 promoter or LIS1 chimeric promoter and operably linked to a coding region of a polynucleotide or selected gene of interest. Enhancer sequences can be found 5′ to the start of transcription in a promoter that functions in eukaryotic cells. Sometimes, these enhancer sequences are found within introns. Enhancer sequences can be inserted in the forward or reverse orientation 5′ or 3′ to the coding sequence of polynucleotide or selected gene of interest. Examples of enhancers which can be used in accordance with the present invention include enhancer sequences from the CaMV 35S RNA promoter and octopine synthase genes (Ellis et al., EMBO J., 6(11):3203-3208 (1987)).

When an enhancer is used in conjunction with a LIS1 promoter or a LIS1 chimeric promoter for the expression of a selected protein, the enhancer is preferably upstream of the promoter and the start codon of the coding region of an operably linked polynucleotide(s) or gene(s) of interest. However, a different arrangement of the enhancer relative to other coding regions of a polynucleotide or selected gene(s) of interest can also be used in order to obtain the beneficial properties conferred by the enhancer. For example, the enhancer can be placed 5′ of the promoter, within the promoter, within the coding region of the polynucleotide(s) or selected gene(s) of interest (including within any other intron sequences that may be present), or 3′ of the coding region.

In addition to enhancer sequences, untranslated leader sequences can also be used in transformation constructs containing the LIS1 promoter or LIS1 chimeric promoter. Preferred leader sequences that can be used in such constructs include those which have sequences that can direct optimum expression of the attached coding region of the operably linked polynucleotide(s) or selected gene(s) of interest (i.e., to include a preferred consensus leader sequence which may increase or maintain mRNA stability, prevent incorrect initiation of translation, or promote more efficient translation initiation). Untranslated leader sequences that can be used in the transformation constructs can be readily determined by those skilled in the art.

The transformation constructs prepared in accordance with the present invention can also contain a 3′ end DNA sequence that acts as a signal for 3′ end processing and allow for the polyadenylation of the mRNA produced by coding sequences operably linked to the LIS1 promoter or LIS1 chimeric promoter. Polyadenylation regions which can be used in conjunction with the LIS1 promoter or LIS1 chimeric promoter, include, but are not limited to, those from a gene encoding the small subunit of a ribulose-1,5-bisphosphate carboxylase-oxygenase (rbcS), the terminator from the nopaline synthase gene of Agrobacterium tumefaciens (nos 3′ end) (Bevan et al., Nucleic Acids Research 11 (2):369-385 (1983)), the terminator for the T7 transcript from the octopine synthase gene of Agrobacterium tumefaciens, and the 3′ end of the protease inhibitor I or II genes from potato or tomato.

The transformation constructs of the present invention can further employ the use of transit peptide and signal sequences. Sequences which are joined to the coding sequence of a polynucleotide or selected gene to be expressed and which may be removed posttranslationally from the initial translation product and that facilitate the transport of a protein into or through intracellular or extracellular membranes, are referred to as transit peptides (these peptides facilitate the transit of protein into vacuoles, vesicles, plastids and other intracellular organelles) and signal sequences (these peptides facilitate transport into the endoplasmic reticulum, golgi apparatus and outside of the cellular membrane) (U.S. Pat. No. 5,728,925 describes a chloroplast transit peptide and U.S. Pat. No. 5,510,471 describes an optimized transit peptide.). By facilitating the transport of the protein into compartments inside and outside the cell, these sequences may increase the accumulation of gene product by protecting them from proteolytic degradation. These sequences also allow for additional mRNA sequences from highly expressed genes to be attached to the coding sequence of the genes. Because mRNA being translated by ribosomes is more stable than non-translatable mRNA, the presence of translatable mRNA preceding the polynucleotide or gene may increase the overall stability of the mRNA transcript from the gene and thereby increase synthesis of the polynucleotide or gene product. Since transit and signal sequences are usually posttranslationally removed from the initial translation product, the use of these sequences allows for the addition of extra translated sequences that may not appear on the final polypeptide. It further is contemplated that targeting of certain proteins may be desirable in order to enhance the stability of the protein (See, U.S. Pat. No. 5,545,818).

The LIS1 promoter or LIS1 chimeric promoter of the present invention can also be used to direct the expression of operably linked screenable marker genes. Examples of coding regions from screenable marker genes that can be used in the present invention include, but are not limited to, those shown in Table 1 below.

TABLE 1

| Gene(s) | Which encodes/allows for |
|---|---|
| beta-glucuronidase (gusA) | enzyme(s) for various chromogenic |
| | substrates |
| beta-lactamase gene¹ | enzyme for various chromogenic substrates |
| beta-galactosidase gene² | enzyme for various chromogenic substrates |
| (β-gal or lacZ) | |
| luciferase (lux) gene³ | for bioluminescence detection |
| aequorin gene⁴ | calcium-sensitive bioluminescence detection |
| gene encoding for | detection of gene expression by ultraviolet |
| green fluorescent protein⁵ | and/or blue light excitiation |

¹Dellaporta et al., In: Chromosome Structure and Function: Impact of New Concepts, 18^{th} Stadler Genetics Symposium, 11: 263-383 (1988).
²Sutcliffe, Proc. Natl. Acad. Sci. USA, 75: 337-3741 (1978).
³Ow et al., Science, 234: 856-859 (1986).
⁴Prasher et al., Biochem. Biophys. Res. Commun., 126(3): 1259-1268 (1985).
⁵Sheen et al., Haseloff et al., Proc. Natl. Acad. Sci. USA, 94(6): 2122-2127 (1997); Reichel et al., Proc. Natl. Acad. Sci. USA, 93(12): 5888-5893 (1996); Tian et al., Plant Cell. Rep., 16: 267-271 (1997); WO 97/41228.

In a further embodiment, the present invention relates to methods and compositions for the efficient expression of selected proteins in plants. The LIS1 promoter or LIS1 chimeric promoter of the present invention can be used to express a selected protein in any type of plant and plant part such as monocotyledonous plants or dicotyledonous plants. Examples of monocotyledonous plants in which the LIS1 promoter or LIS1 chimeric promoter can be used include, but are not limited to: Amaryllidaceae (Allium, Narcissus); Graminae, alternatively Poaceae, (Avena, Horedum, Oryza, Panicum, Pennisetum, Poa, Saccharum, Secale, Sorghum, Triticum, Zea). Examples of dicotyledonous plants in which the LIS1 promoter or LIS1 chimeric promoter can be used include, but are not limited to: Apocynaceae (Catharanthus); Asteraceae, alternatively Compositae (Aster, Calendula, Callistephus, Cichorium, Coreopsis, Dahlia, Dendranthema, Gazania, Gerbera, Helianthus, Helichrysum, Lactuca, Rudbeckia, Tagetes, Zinnia); Balsaminaceae (Impatiens); Begoniaceae (Begonia); Caryophyllaceae (Dianthus); Chenopodiaceae (Beta, Spinacia); Cucurbitaceae (Citrullus, Curcurbita, Cucumis); Cruciferae (Alyssum, Brassica, Erysimum, Matthiola, Raphanus); Gentinaceae (Eustoma); Geraniaceae (Pelargonium); Euphorbiaceae (Poinsettia); Labiatae (Salvia); Leguminosae (Glycine, Lathyrus, Medicago, Phaseolus, Pisum); Liliaceae (Lilium); Lobeliaceae (Lobelia); Malvaceae (Abelmoschus, Gossypium, Malva); Plumbaginaceae (Limonium); Polemoniaceae (Phlox); Primulaceae (Cyclamen); Ranunculaceae (Aconitum, Anemone, Aquilegia, Caltha, Delphinium, Ranunculus); Rosaceae (Rosa); Rubiaceae (Pentas); Scrophulariaceae (Angelonia, Antirrhinum, Torenia); Solanaceae (Capsicum, Lycopersicon, Nicotiana, Petunia, Solanum); Umbelliferae (Apium, Daucus, Pastinaca); Verbenaceae (Verbena, Lantana); Violaceae (Viola).

As mentioned briefly previously, the present invention provides a LIS1 promoter and LIS1 chimeric promoter for the expression of selected proteins in plants and plant parts. The choice of a selected protein for expression in a plant host cell in accordance with the invention will depend on the purpose of the transformation. One of the major purposes of transformation of crop plants is to add commercially desirable, agronomically or horticulturally important traits to the plant. Such traits include, but are not limited to, insect resistance or tolerance; disease resistance or tolerance (viral, bacterial, fungal), color (anthocyanins (such as, but not limited to, pelargonidin, cyanidin, delphinidin, peonidin, malvidin, petunidin and the like) and/or carotenoids (such as, but not limited to, phytoene, phytofluene, ζ-carotene, neurosporene, lycopene, γ-carotene, β-carotene, α-cryptoxanthin, β-cryptoxanthin, canthaxanthin, capsanthin, capsorubin, zeaxanthin, violaxanthin, neoxanthin, antheraxanthin, lutein, astaxanthin and the like)), floral scent, flower longevity and the like.

In a further embodiment of the present invention, transformation of a recipient plant cell may be carried out with more than one polynucleotide and/or selected gene of interest. Two or more exogenous coding regions from one or more polynucleotides or selected genes of interest also can be supplied in a single transformation event using either distinct transgene-encoding vectors, or using a single vector incorporating two or more coding sequences. For example, plasmids bearing the bar and aroA expression units in either convergent, divergent, or colinear orientation, are considered to be particularly useful. Any two or more transgenes of any description, such as those conferring insect, disease (viral, bacterial, fungal) or color (anthocyanins (such as, but not limited to, pelargonidin, cyanidin, delphinidin, peonidin, petunidin and the like) and/or carotenoids (such as, but not limited to, phytoene, phytofluene, ζ-carotene, neurosporene, lycopene, y-carotene, P-carotene, α-cryptoxanthin, β-cryptoxanthin, canthaxanthin, capsanthin, capsorubin, zeaxanthin, violaxanthin, neoxanthin, antheraxanthin, lutein, astaxanthin and the like) floral scent, or flower longevity may be employed as desired.

In another embodiment, LIS1 promoter or LIS1 chimeric promoter of the present invention can be employed for the purpose of introducing an operably linked polynucleotide(s) or selected gene(s) into plants for the purpose of expressing RNA molecules (transcripts) that affect plant phenotype but which are not translated into protein. Such a purpose can be affected through the use of antisense RNA, RNA enzymes called ribozymes, or though the production of RNA transcripts that are capable of promoting RNase P-mediated cleavage of target mRNA molecules. Antisense RNA, ribozymes or RNase P-mediated cleavage of target mRNA can be used to reduce or eliminate expression of native or introduced plant genes in a transformed plant.

The expression of at least one antisense RNA molecule can be used to suppress the expression of a target molecule, using routine techniques known in the art. More specifically, the present invention contemplates the construction of an expression cassette in which the promoter of the present invention can be operably linked to a polynucleotide sequence that encodes a complementary polynucleotide unit (such as an antisense RNA molecule). The binding of this complementary polynucleotide unit to a target molecule can be inhibitory. For example, if the target molecule is an mRNA molecule, the binding of RNA, the complementary polynucleotide unit, results in hybridization and in an arrest of translation of a target protein.

Alternatively, the promoter of the present invention can be operably linked to a polynucleotide sequence that encodes a ribozyme. More specifically, the present invention contemplates the construction of an expression vector in which the promoter of the present invention is operatively linked to a polynucleotide sequence that encodes a ribozyme. It is known in the art that ribozymes can be designed to express endonuclease activity directed to a certain target sequence in a mRNA molecule. For example, up to 100% inhibition of neomycin phosphotransferase gene expression was achieved by ribozymes in tobacco protoplasts (See, Steinecke et al., EMBO J., 11:1525 (1992)). In the present invention, examples of appropriate target RNA molecules for ribozymes include mRNA species that encode for biosynthetic enzymes found in plant biochemical pathways.

In yet a further alternative, the promoter of the present invention can be used to direct the production of RNA molecules (transcripts) that are capable of promoting RNase P-mediated cleavage of target mRNA molecules. More specifically, the present invention further contemplates the construction of an expression vector in which the promoter of the present invention directs the production of RNA transcripts that are capable of promoting RNase P-mediated cleavage of target mRNA molecules. Under this approach, an external guide sequence can be constructed for directing the endogenous ribozyme, RNase P, to a particular species of mRNA, which is subsequently cleaved by the cellular ribozyme (See, U.S. Pat. No. 5,168,053; Yuan et al., Science, 263:1269 (1994)).

The present invention further contemplates that the LIS1 promoter or LIS1 chimeric promoter can be used to introduce at least one polynucleotide(s) or selected gene(s) to produce transgenic plants having reduced expression of a native gene product via the mechanism of co-suppression. As shown in tobacco, tomato and petunia (Goring et al., Proc. Natl. Acad. Sci. USA, 88:1770-1774 (1991), Smith et al., Mol. Gen. Genet., 224:447-481 (1990), Napoli et al., Plant Cell, 2:279-289 (1990), van der Krol et al., Plant Cell, 2:291-299 (1990)), expression of a sense transcript of a native gene can reduce or eliminate expression of a native gene in a manner similar to that observed for antisense genes. The gene introduced can encode all or part of the targeted native protein; however, its translation may not be required for reduction of levels of native protein.

The present invention further contemplates the use of one or more assays known in the art in order to ascertain or determine the efficiency of transgene expression. For example, assays could be used to determine the efficacy of the LIS1 promoter or LIS1 chimeric promoter in directing protein expression in at least one flower in a plant when used in conjunction with various different enhancer sequences, terminators or other regulatory elements. Also, assays could be used to determine the efficacy of various deletion mutants of the LIS1 promoter or LIS1 chimeric promoter in directing the expression of proteins.

The biological sample to be assayed can be polynucleotides isolated from the cells of any plant material using molecular biology techniques known in the art (Sambrook et al., In: Molecular Cloning: A Laboratory Manual, Second edition, Cold Spring Harbor Laboratory, Cold Spring Harbor, N.Y., 1989). The polynucleotide can be genomic DNA or fractionated or whole cell RNA. Where RNA is used, it can be converted to DNA if appropriate to do so.

Examples of various assays that can be used in the present invention include fluorescent in situ hybridization (“FISH”), direct DNA sequencing, pulsed field gel electrophoresis (“PFGE”) analysis, RNA or DNA gel blot analysis, single-stranded conformation analysis (“SSCA”), RNase protection assay, allele-specific oligonucleotide (“ASO”), dot blot analysis, denaturing gradient gel electrophoresis and restriction fragment polymorphism (“RFLP”).

In order to determine the efficiency with which a particular transgene is expressed is to purify and quantify a polypeptide expressed by the transgene. Techniques for purifying proteins are well known in the art. These techniques include, but are not limited to, ion-exchange chromatography, affinity chromatography, exclusion chromatography, gel electrophoresis, isoelectric focusing, fast protein liquid chromatography or high performance liquid chromatography. In addition, immunological procedures can be used for protein detection. Methods include, but are not limited to, enzyme-linked immunosorbent assay (“ELISA”), Western blot and radioimmunoassay (“RIA”).

Suitable methods for plant transformation for use in connection with the present invention include any method by which DNA can be introduced into a host cell, including, but not limited to, the methods described below in Table 2.

TABLE 2

| Method for Plant | |
|---|---|
| Transformation | Reference |
| Direct delivery of DNA | Omirulleh et al., Plant Mol. Biol., |
| (i.e. PEG-mediated | 21(3): 415-428 (1993). |
| transformation of | |
| protoplasts or calcium | |
| phosphate precipitation). | |
| Desiccation/inhibition- | Potrykus et al., Mol. Gen. Genet., 199: |
| mediated DNA uptake | 183-188 (1985). |
| Electroporation | U.S. Pat. No. 5,384,253, Fromm et al., |
| | Proc. Natl. Acad. Sci. USA 82: 5824 (1985). |
| Agrobacterium-mediated | U.S. Pat. Nos. 5,591,616 |
| transformation | and 5,563,055, Horsch et al., Science |
| | 233: 496-498 (1984), and Fraley et al., Proc. |
| | Natl. Acad. Sci. USA 80: 4803 (1983). |
| | Although Agrobacterium is useful primarily |
| | in dicots, certain monocots can be |
| | transformed by Agrobacterium. For |
| | instance, Agrobacterium transformation of |
| | rice is described by Hiei et al., Plant J., |
| | 6: 271-282 (1994). |
| Microprojectile | U.S. Pat. Nos. 5,550,318, 5,538,880, |
| bombardment | 5,610,042 and W0 94/09699. |

Plant cells transformed by any of the above transformation techniques can be cultured to regenerate a whole plant that possesses the transformed genotype. Such regeneration techniques rely on manipulation of certain phytohormones in a tissue culture growth medium, typically relying on the marker gene, which has been introduced together with the LIS1 promoter or LIS1 chimeric promoter and the coding region from the polynucleotide or selected gene of interest. Plant regeneration from cultured protoplasts is described in Evans et al., Protoplasts Isolation and Culture, Handbook of Plant Cell Culture, pp. 124-176, Macmillan Publishing Company, New York, 1983; and Binding; Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al., Ann. Ref. of Plant Phys. 38:467-486 (1987).

The methods of the present invention are particularly useful for incorporating various polynucleotides or selected genes of interest into transformed plants in ways and under circumstances that are not found naturally. In particular, various polynucleotides or selected genes can be expressed at times or in quantities that are not characteristic of natural plants.

One skilled in the art will recognize that after the transformation construct is stably incorporated in transgenic plants and confirmed to be operable, it can be introduced into other plants by sexual crossing. Any of a number of standard breeding techniques can be used, depending upon the species to be crossed.

By way of example, and not of limitation, examples of the present invention will now be given.

### EXAMPLE 1

### pBHX Plasmids

pBHX109

A 2.4 Kb Hind III—EcoR I fragment consisting of the promoter-containing region of the Arabidospis UBQ3 polyubiquitin gene (1.3 kb) fused to the GFP gene (the sm-RSGFP version contained within plasmid pCD3-327 that is available from the Arabidopsis Biological Resource Center in Columbus, Ohio) and nos polyA signal-containing region was isolated. This fragment was then ligated into a T-DNA binary vector previously digested with Hind III and EcoR I to create pBHX109.

pBHX113

A Hind III—EcoR I fragment consisting of the promoter-containing region of the Clarkia brewerii LIS1 gene (the same region found in pBHX103) fused to the GFP gene (the sm-RSGFP version contained within plasmid pCD3-327 that is available from the Arabidopsis Biological Resource Center in Columbus, Ohio) and nos polyA signal-containing region was isolated. This fragment was then ligated into a T-DNA binary vector previously digested with Hind III and EcoR I to create pBHX 13.

pBHX94

A plasmid containing the 5′-flanking region of the Clarkia brewerii LIS1 gene was obtained from the University of Michigan. A ˜1 kb fragment containing the LIS1 5′-flanking region was synthesized by PCR using the primers: BHX30: CCAAGCTTATCTAATAATGTATCAAAATC (SEQ ID NO: 3) and BHX31: GGCCATGGTTGTCTTGTTTAAGGTGG (SEQ ID NO: 5). These primers were designed to anneal to the 5′ flanking region at one end and within the 5′ untranslated leader region at the 3′ end. The PCR product was digested with the restriction enzymes, Hind III and Nco I, which cleave at the 5′ and 3′ ends of the fragment, respectively. The Nco I site overlaps the initiation codon of the LIS1 protein-coding region. The digested fragment was gel-purified and subsequently fused in-frame to a plasmid-bome, promoterless gusA::nos transgene (previously digested with Hind III and Nco I) to create a LIS1::gusA::nos transgene (designated pBHX94) pBHX99 Plasmid pBHX94 was digested with Hind III and EcoR I to liberate a fragment containing the LIS1::gusA::nos transgene. This fragment was then ligated into a T-DNA binary vector previously digested with Hind III and EcoR I to create pBHX99.

pBHX103

A plasmid containing the 5′-flanking region of the Clarkia brewerii LIS1 gene was obtained from the University of Michigan. A 1 kb fragment containing the LIS1 5′-flanking region was synthesized by PCR using the primers: BHX30: CCAAGCTTATCTAATAATGTATCAAAATC (SEQ ID NO: 3) and BHX36: CAGCCCGGGATGGTTGTCTTGTTTAAGGTGG (SEQ ID NO:4). These primers were designed to anneal to the 5′ flanking region at one end and within the 5′ untranslated leader region at the 3′ end. The PCR product was digested with the restriction enzymes, Hind III and Sma I, which cleave at the 5′ and 3′ ends of the fragment, respectively. The digested fragment was gel-purified and subsequently inserted into a Hind III- and Sma I-digested plasmid containing a multi-cloning site region (MCS) followed by the nos polyA signal-containing region to create a LIS1::MCS::nos transgene (designated pBHX103).

pBHX107

A 1.5 kb Hinc II fragment from plasmid pATC921 (containing the crtB gene with a rbsS transit peptide fused to the N-terminus of the crtB protein-coding region) was inserted in the sense orientation into the Sma I site located between the LIS1 promoter and the nos fragments of plasmid pBHX103 to create plasmid pBHX107.

pBHX112

Plasmid pBHX107 was digested with Hind III and EcoR I to liberate a fragment containing the LIS1::crtB::nos transgene. This fragment was then ligated into a T-DNA binary vector previously digested with Hind III and EcoR I to create pBHX112.

### EXAMPLE 2

### Evaluation of Gene Expression in Transgenic Petunia Lines Transformed with Plasmids Containing either the LIS1 Promoter or the Constitutive UBQ3 Promoter.

To evaluate gene expression in flower tissue using the LIS1 promoter, ‘Mitchell’ petunia was transformed with either LIS1::GFP::nos (pBHX113) or UBQ3::GFP::nos (pBHX109) constructs. UBQ3 is a constitutive promoter well known in the art. Petunia transformants were generated. Once flowering plants were established in the greenhouse, sample flowers from each plant were evaluated for GFP expression using blue light generated from a fluorescent microscope. A subjective rating system, 0 indicating no visible expression up to 4 representing the highest expression, was used.

The results are shown below in Table 3. LIS1 directed GFP expression was most evident in the petal and throat flower tissue. Of the LIS1 and UBQ3 transgenic plants tested, only two LIS1 promoter-containing plants had GFP expression in the pistil tissue. UBQ3 directed GFP expression was more uniform throughout the flower tissue, as would be expected with a constitutive-type promoter. Results demonstrate comparable GFP expression between the LIS1 promoter and the UBQ3 constitutive promoter in the petal tissue.

TABLE 3
GFP Expression

| Line | Petal | Throat | Pistil | Stamens | Nectary |
|---|---|---|---|---|---|
| LIS1::GFP::nos | | | | | |
| 113A-4500-3-1 | 2 | 2 | 3 | 0 | 2 |
| 113A-4500-3-4 | 4 | 4 | 0 | 3 | 0 |
| 113A-4500-3-5 | 2 | 2 | 0 | 0 | 0 |
| 113A-4500-3-6 | 1 | 1 | 0 | 0 | 0 |
| 113A-4500-3-7 | 3 | 3 | 0 | 0 | 0 |
| 113A-4500-3-8 | 0 | 1 | 0 | 0 | 0 |
| 113A-4500-3-9 | 0 | 2 | 0 | 0 | 0 |
| 113A-4500-3-10 | 0 | 4 | 0 | 0 | 0 |
| 113A-4500-3-11 | 3 | 3 | 0 | 0 | 0 |
| 113A-4500-3-14 | 0 | 0 | 2 | 0 | 0 |
| 113A-4500-3-15 | 0 | 0 | 0 | 0 | 0 |
| 113A-4500-3-16 | 0 | 3 | 0 | 0 | 0 |
| 113A-4500-3-17 | 4 | 4 | 0 | 3 | 0 |
| 113A-4500-3-20 | 4 | 4 | 0 | 2 | 0 |
| UBQ3::GFP::nos | | | | | |
| 109A-4400-2-21 | 3 | 3 | 0 | 4 | 3 |
| 109A-4400-2-22 | 0 | 2 | 0 | 0 | 0 |
| 109A-4400-2-29 | 4 | 4 | 0 | 4 | 4 |
| 109A-4400-2-30 | 0 | 2 | 0 | 0 | 0 |
| 109A-4400-2-31 | 1 | 1 | 0 | 0 | 0 |
| 109A-4400-2-34 | 0 | 0 | 0 | 3 | 0 |
| 109A-4400-2-35 | 4 | 4 | 0 | 4 | 3 |
| 109A-4400-2-36 | 3 | 3 | 0 | 0 | 0 |
| 109A-4400-2-37 | 0 | 0 | 0 | 0 | 0 |
| 109A-4400-2-38 | 0 | 0 | 0 | 3 | 3 |
| 109A-4400-2-39 | 0 | 2 | 0 | 3 | 0 |
| 109A-4400-2-42 | 3 | 3 | 0 | 4 | 4 |
| 109A-4400-2-43 | 4 | 4 | 0 | 0 | 0 |
| 109A-4400-2-45 | 0 | 0 | 0 | 2 | 0 |

### EXAMPLE 3

### Evaluation of Gene Expression in Transgenic Petunia Lines Transformed with Plasmids Containing either the LIS1 Promoter or 35S Promoter from CaMV

Eleven (11) transgenic ‘Dreams White’ petunia lines (‘Dreams White’ commercially available from PanAmerican Seed Company, 622 Town Road, West Chicago, Ill.) containing the plasmid LIS1::gusA::nos (pBHX99) and three transgenic lines containing the 35S::gusA::nos construct (pBI121) were generated. Once flowering plants were established in the greenhouse, sample flowers and young leaves from each plant were histochemically stained with a 5-bromo-4-chloro-3-indolyl-beta-D-glucuronic acid, cyclohexylammonium salt solution (X-gluc) and evaluated for GUS expression in various tissues (See Table 4 below). A subjective rating system, 0 indicating no visible expression up to 3 representing the highest expression, was used.

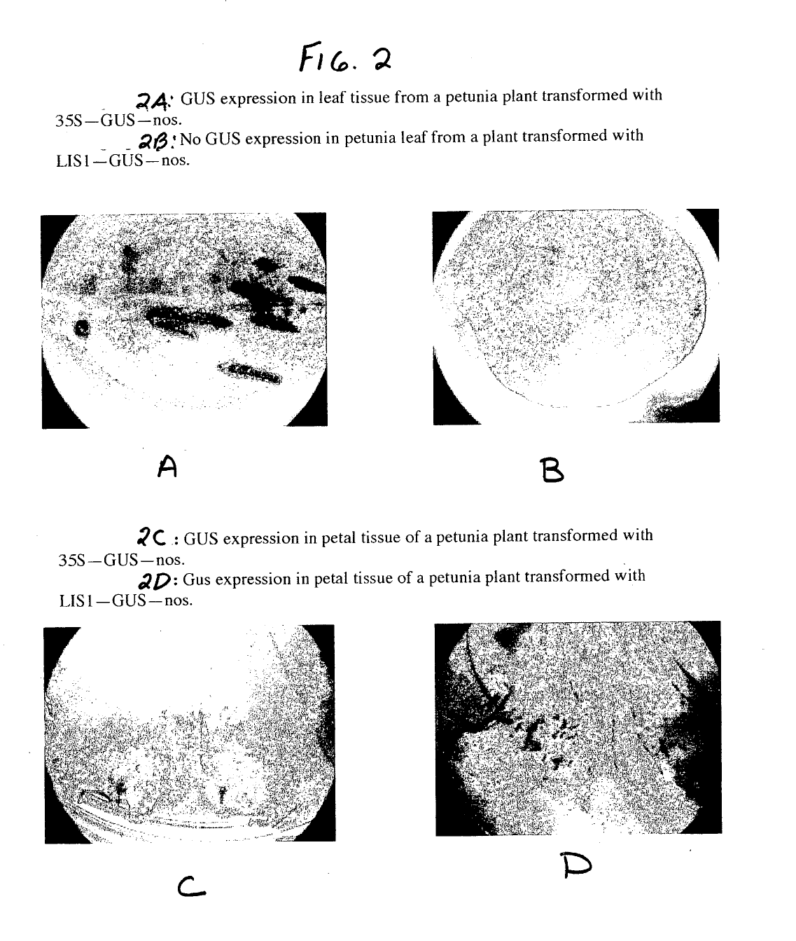

As shown in Table 4 below, several lines demonstrate flower-preferred expression. In contrast, all three (3) of the 35S::gusA::nos (pBI121) lines demonstrated moderate to high GUS expression in all tissues tested (See Table 4 below). The results demonstrate that some of the LIS1::gusA::nos (pBHX99) transgenic lines exhibited GUS expression in leaf tissues, indicating that the LIS1 promotercan be improperly expressed after integration into particular regions of the petunia genome (so-called ‘position effect’). However, most of the (six (6)) LIS1::gusA::nos (pBHX99) lines identified showed moderate to high expression in petal and other flower tissues while having no visible GUS expression in the leaves. The photographs shown in FIG. 2 demonstrate the differences in GUS expression between transgenic petunia lines containing LIS1::gusA::nos or 35S::gusA::nos (pBI121) constructs.

TABLE 4
GUS Expression

| Line | Petal | Anthers/Pollen | Pistil/Nectary | Leaf |
|---|---|---|---|---|
| LIS1::gusA::nos | | | | |
| PET-1700-1-2 | 2 | 2 | NA* | 0 |
| PET-1700-1-4A | 2 | 1 | 3 | 0 |
| PET-1700-1-6 | 2 | 2 | NA | 0 |
| PET-1700-1-7A | 2 | 3 | 3 | 1 |
| PET-1700-1-10 | 1 | 1 | NA | 0 |
| PET-1700-1-13B | 2 | 2 | NA | 2 |
| PET-1700-1-14A | 2 | 3 | 2 | 1 |
| PET-1700-1-17 | 3 | 3 | 2 | 1 |
| PET-1700-1-21A | 2 | 2 | 3 | 0 |
| PET-1700-1-22B | 2 | 1 | 3 | 3 |
| PET-1700-1-26A | 2 | 1 | 2 | 0 |
| 35S::gusA::nos | | | | |
| PET-1500-1-1A | 2 | 2 | 1 | 3 |
| PET-1500-1-6A | 3 | 3 | 2 | 3 |
| PET-1500-1-15A | 1 | 3 | 3 | 3 |

*Not Assayed

To gain a quantitative measure of GUS activity in the LIS1::gusA::nos (pBHX99) transformed plants compared to 35S::gusA::nos (pBI121) transformants, fluorometric detection of GUS expression utilizing cell-free crude extracts from various plant tissues was carried out using methylumbelliferyl-B-D-glucuronide (MUG) as the substrate. Duplicate analyses were performed, and the results are shown below in Table 5.

All LIS1::gusA::nos transformed plants demonstrated GUS expression in petals that was greater than the control, and with the exception of one plant (1700-1-6), was greater than any of the petals of the 35S::gusA::nos transformed plants. In addition, the GUS expression in petals was significantly higher than leaf and calyx tissues for all LIS1::gusA::nos transformed plants. Petunia line 1700-1-14A exhibited the highest expression in petal tissue having levels of 2160.4+/−626.3 nmol/min/g[fw]. This level of GUS activity is more than 28-fold higher than the CaMV 35S RNA promoter, a promoter known to be very active in dicotyledonous plants. Thus the LIS1 promoter can be concluded to direct high levels of expression in petunia petals.

TABLE 5
GUS Expression
nmol/min/g[fw] +/− standard deviation

| Line | Petal | Leaf | Calyx |
|---|---|---|---|
| Control | 0.1 +/− 0.4 | 0.0 +/− 0.2 | 0.0 +/− 0.2 |
| ‘Dreams White’ | | | |
| LISI::gusA::nos | | | |
| 1700-1-2 | 219.7 +/− 54.9 | 1.0 +/− 1.3 | 1.5 +/− 1.9 |
| 1700-1-4A | 90.0 +/− 8.8 | 1.0 +/− 1.6 | 2.6 +/− 3.1 |
| 1700-1-6 | 54.8 +/− 20.1 | 0.3 +/− 0.1 | 1.0 +/− 1.4 |
| 1700-1-7A | 246.0 +/− 107.1 | 29.5 +/− 7.9 | 46.7 +/− 28.3 |
| 1700-1-10 | 153.8 +/− 95.7 | 0.2 +/− 0.7 | 0.3 +/− 0.1 |
| 1700-1-12A | 352 +/− 39.7 | 0.3 +/− 0.4 | 0.2 +/− 1.0 |
| 1700-1-13B | 812.5 +/− 40.8 | 44.1 +/− 21.9 | 36.1 +/− 17.6 |
| 1700-1-14A | 2160.4 +/− 626.3 | 18.1 +/− 9.6 | 29.4 +/− 26.5 |
| 1700-1-17 | 297.0 +/− 289.8 | 3.3 +/− 4.3 | 1.9 +/− 1.0 |
| 1700-1-21A | 625.2 +/− 127.5 | 0.4 +/− 0.4 | 1.6 +/− 2.5 |
| 1700-1-22B | 1626.5 +/− 746.4 | 12.3 +/− 6.0 | 40.6 +/− 46.9 |
| 1700-1-26A | 91.1 +/− 12.1 | 5.2 +/− 2.9 | 4.8 +/− 0.7 |
| 35S::gusA::nos | | | |
| 1500-1-1A | 44.4 +/− 2.1 | 67.0 +/− 22.1 | 46.6 +/− 36.3 |
| 1500-1-6A | 72.4 +/− 9.6 | 124.8 +/− 4.1 | 94.3 +/− 43.0 |
| 1500-1-15A | 75.3 +/− 4.0 | 111.1 +/− 4.3 | 92.6 +/− 25.9 |

To illustrate that the LIS1 promoter provides flower preferred gene expression in other petunia varieties, GUS activity was measured in transformants of ‘Mitchell’ petunia. Duplicate analyses were performed, and the results are shown below in Table 6. Although overall GUS expression is lower in petals of LIS1::gusA::nos ‘Mitchell’ petunia compared to petals of transgenic ‘Dreams White’ the trends are the same. GUS expression levels in petals of LIS1::gusA::nos transgenic ‘Mitchell’ petunia ranged from 61 to 419 nmol/min/g[fw] while petals of the 35S::gusA::nos line reached a level of 63 nmol/min/g[fw] demonstrating that levels of gene expression in petals of LIS1::gusA::nos transgenic ‘Mitchell’ petunia are as high or higher (up to six-fold higher for line 99A-2700-1-5) than for the 35S::gusA::nos line. In contrast, the level of GUS expression in leaf and calyx tissues ranged from 0.7 to 7.3 and 1.9 to 20.5 nmol/min/g[fw] respectively in LIS1::gusA::nos transgenic lines. GUS expression in the 35S::gusA::nos transgenic line reached 104 nmol/min/g[fw] in leaf tissue and 93.9 nmol/min/g[fw] in calyx tissue. The uniformity of GUS expression in different tissues of the 35S::gusA::nos transgenic line is to be expected, since 35S is a known constitutive promoter. The contrasting results obtained in the LIS1::gusA::nos transgenic lines, both ‘Dreams White’ and ‘Mitchell’ petunias, clearly show flower preferential expression with the LIS1 promoter.

Thus, the histochemical and fluorometric assay results for GUS activity are consistent and demonstrate that the LIS1 promoter is able to direct high levels of transgene expression in the floral tissues of petunia.

TABLE 6
GUS Expression
nmol/min/g[fw] +/− standard deviation

| Line | Petal | Leaf | Calyx |
|---|---|---|---|
| Control ‘Mitchell’ | −1.7 +/− 2.7 | 0.1 +/− 0.0 | 0.1 +/− 0.3 |
| LISI::gusA::nos | | | |
| 99A-2700-1-10 | 419.9 +/− 64.3 | 7.3 +/− 3.1 | 20.5 +/− 18.0 |
| 99A-2700-1-5 | 325.3 +/− 147.1 | 0.5 +/− 1.3 | 5.0 +/− 2.3 |
| 99A-2700-1-8 | 72.3 +/− 0.3 | 0.5 +/− 0.3 | 1.3 +/− 0.2 |
| 99A-2700-1-2 | 61.0 +/− 4.3 | 0.7 +/− 0.1 | 1.9 +/− 0.7 |
| 35S::gusA::nos | | | |
| 1500-1-15A | 63.0 +/− 22.6 | 104.0 +/− 34.9 | 93.9 +/− 35.7 |

### EXAMPLE 4

### Evaluation of Gene Expression in Flower Parts and Developing Petals of a Transgenic Petunia Transformed with Plasmid Containing the LIS1 Promoter

Transgenic ‘Dreams White’ petunia designated 1700-1-14A containing the plasmid LIS1::gusA::nos was identified as the line with the highest GUS expression in the petal tissue (See Table 5 above). This line was further used in studies to examine the effects of flower age, as determined by bud or flower length, on GUS expression and to determine GUS expression in specific flower parts of mature flowers.

GUS activity in petals was determined for 1700-1-14A flowers selected from 2 to 5 cm in length as measured from the calyx to petal edge. A closed-bud stage is typically 2 cm while 5 cm is a fully open flower. Results, shown in Table 7 below, reveal a 174-fold increase in GUS expression from bud to mature flower, indicating that as flowers matured, GUS expression increased.

TABLE 7

| | GUS Expression |
|---|---|
| Flower Size | nmol/min/g[fw] |
| 2 cm | 21 |
| 3 cm | 84 |
| 4 cm | 1944 |
| 5 cm | 3655 |
| 5 cm | 1322 |
| 5 cm | 2366 |

GUS activity in flower parts was determined for 1700-1-14A fully open flowers. Flower parts examined included petal top (distal), petal base, anther/pollen, and pistil. Duplicate analyses were performed, and the results are shown below in Table 8. GUS expression was observed in all flower tissues with the highest GUS expression being in the pistils. Pistil GUS expression was 14 fold higher than the distal petal tissue. Thus, the LIS1 promoter can be concluded to direct high levels of expression throughout petunia flower tissues.

TABLE 8

| | GUS Expression |
|---|---|
| Flower Tissue | nmol/min/g[fw] |
| Petal top | 2282 +/− 689 |
| Petal base | 964 +/− 208 |
| Anther/Pollen | 5369 +/− 613 |
| Pistil | 33402 +/− 14790 |

### EXAMPLE 5

### Evaluation of Gene Expression in Transgenic Marigold Lines Transformed with a Plasmid Containing the LIS1 Promoter

To determine whether the LIS1 promoter was able to direct high levels of GUS expression in a second heterologous plant species, a marigold (Tagetes erecta) plant PanAmerican Seed proprietary breeding line 13819 was transformed with the LIS1::gusA::nos (pBHX99) construct and one transformant identified as line 99A-3300-1-10 was recovered. Fluorometric assays to detect GUS expression were carried out on different plant tissues of this line. As was found for LIS1::gusA-expressing petunias, GUS expression (nmol/min/g[fw]) was high in the floral tissues, but not in the calyx and leaf. These results indicate that LIS1 promoter directed flower-preferred gene expression in marigold.

Additional transgenic plants of PAS breeding line13819, transformed with LIS1::gusA::nos (pBHX99), were evaluated using fluorometric assays to detect GUS activity. Also evaluated were progeny from the line 99A-3300-1-10 noted above crossed with a control male parent PanAmerican Seed proprietary breeding line 13819. Duplicate analyses were performed, and the results are shown below in Table 9. Relatively high levels of LIS1-directed GUS expression were observed in marigold flower tissue including the pistils, petals and developing seeds. GUS expression up to 351 nmol/min/g [fw] was observed in the pistils. This level is comparable to the LIS1-directed GUS expression observed in petal tissue of ‘Mitchell’ petunia. Much lower or no expression was observed in the leaf tissue. In two of the six receptacles tested, high-level expression was observed.

Analysis of the progeny, 99A-10-2, 99A-10-3, 99A-10-17 and 99A-10-18, from a cross using transgenic 99A-3300-1-10, demonstrates that the transgene is sexually inheritable. In addition, two of the progeny (99A-10-17 and 99A-10-18) were among the highest petal expression levels observed.

The level of LIS1-directed GUS expression in flower tissue is higher than that observed in the flowers of control plants, and with the exception of three lines tested, LIS1-directed GUS expression in flower tissue is higher than the 35S::gusA::nos transformed plants. Thus, the LIS1 promoter can be concluded to be inheritable in marigold and to direct high levels of transgene expression in marigold as well as petunia flower tissue.

TABLE 9
GUS Expression
nmol/min/g[fw]

| Line | Petal | Leaf | Seed | Receptacle |
|---|---|---|---|---|
| Control PAS | −0.1 | 0.0 | | |
| LISI::gusA::nos | | | | |
| 99A-0601-1-3 | 7.4 | −0.5 | | |
| 99A-0701-2-1 | 16.4 | 6.8 | | |
| 99A-0701-2-6 | 37.5 | 14.9 | | |
| 99A-0701-2-9 | 2.4 | 0.6 | | |
| 99A-0701-2-12 | 17.0 | 3.0 | 28.2 | 47.6 |
| 99A-1401-2-4 | 0.8 | 0.1 | | |
| 99A-1401-2-7 | 0.9 | 0.1 | | |
| 99A-1401-2-8 | 0.5 | 0.0 | | |
| 99A3300-1-7 | 3.1 | −0.9 | | |
| 99A-10-17 | 18.1 | 1.7 | 30.4 | 31.5 |
| 99A-10-18 | 32.4 | 1.8 | 42.0 | 1.6 |
| 35S::gusA::nos | | | | |
| 3301-2101-1-2 | 1.1 | 29.0 | | −0.6 |

### EXAMPLE 6

### Evaluation of Transgenic Petunia Lines Transformed with a Plasmid Containing the LIS1 Promoter and crtB

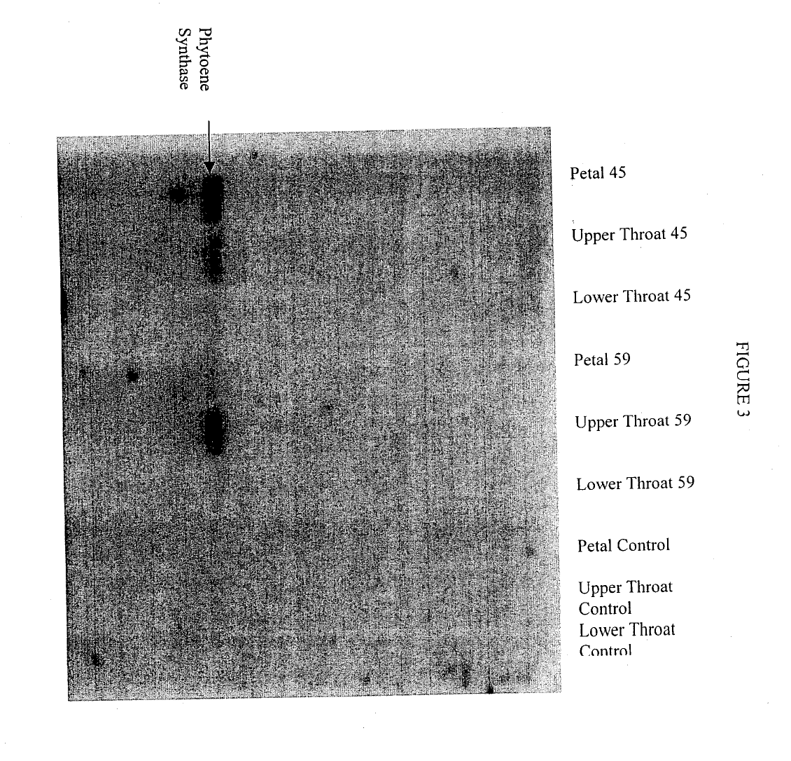

Flowers from two ‘Mitchell’ petunia plants transformed with pBHX112 containing LIS1::crtB::nos were analyzed to determine if gene expression was localized to a particular flower sector. The crtB gene encodes for the enzyme phytoene synthase., an enzyme that produces phytoene, a colorless carotenoid intermediate found early in the carotenoid biosynthetic pathway. Plants were identified as 112-3200-1-45 and 112-3200-1-59, and flowers were segmented into three parts: petal, upper throat and lower throat. From each part total RNA was extracted and an RNA gel blot analysis was performed. Results shown in FIG. 3 indicate that crtB mRNA is present in moderate levels in both petal and upper throat tissue and to a lesser extent in lower throat tissue, confirming that gene expression can be detected at the transcript level.

To further demonstrate gene activity in petal tissue, HPLC analysis was performed using ‘Mitchell’ petunia lines transformed using LIS1::crtB::nos (pBHX112) construct. For HPLC analysis the petal tissue extraction procedure followed the official method for extraction of carotenes and xanthophylls in dried plant material (See, Official Methods of Analysis (1980) 13^{th} Ed., AOAC, Arlington, Va., sec. 43.018-43.023). Tissue was not saponified during extraction.

HPLC equipment comprised an Alliance 2690 equipped with a refrigerated autosampler, column heater and a Waters Photodiode Array 996 detector (Waters Corp., Milford, Mass.). Separation was obtained with a YMC C30 column, 3 μm, 2.0×150 mm, with a guard column of the same material. Standards were obtained from ICC Indofine Chemicals, Somerville, N.J., and from DHI-Water & Environment, Horsholm, Denmark. The dried samples were resuspended in methyl tert-butyl ether and methanol to a total volume of 200 microliters and filtered. Carotenoids were separated using a gradient method. Initial gradient conditions were 90% methanol: 5% water: 5% methyl tert-butyl ether at a flow rate of 0.4 milliliters per minute. From zero to 15 minutes the mobile phase was changed from the initial conditions to 80 methanol: 5 water: 15 methyl tert-butyl ether, and from 15 to 60 minutes to 20 methanol: 5 water: 75 methyl tert-butyl ether. For the following 10 minutes, the mobile phase was returned to the initial conditions and the column equilibrated for an additional 15 minutes. The column temperature was maintained at 27° C. Injections were 10 μL. Values for carotenoids shown in Table 10 below are indicated using peak area as percent of the total area at 450 nm. Phytoene was identified based on spectral signature, and phytoene area was determined from a max plot. Data is expressed as normalized peak area and numbers in parentheses represent the percent each carotenoid contributes to the total carotenoid peak areas.

LIS1 promoter activity in the petal tissue is evident in an observed over 10-fold increase in β-carotene levels as compared to the control. Also observed in the transgenics was the presence of phytoene, which was undetected in control petal tissue. Zeaxanthin and lutein contents are not significantly different from controls.

TABLE 10

| | Peak Area | | | |
|---|---|---|---|---|
| Line | β-carotene | phytoene | zeaxanthin | lutein |
| 112A-3200-1-29 | 3962 (48) | 385 (4) | 47 (1) | 1603 (20) |
| 112A-3200-1-31 | 3830 (46) | 2500 (18) | 0 (0) | 1765 (21) |
| 112A-3200-1-43 | 2749 (24) | 375 (2) | 337 (3) | 2939 (26) |
| 112A-3200-1-41 | 2492 (52) | 1536 (18) | 126 (3) | 1168 (24) |
| 112A-3200-1-53 | 2483 (36) | 2159 (17) | 158 (2) | 1557 (23) |
| 112A-3200-1-45 | 2350 (39) | 1331 (15) | 118 (2) | 1701 (28) |
| 112A-3200-1-25 | 2202 (39) | 453 (5) | 191 (3) | 1226 (21) |
| 112A-3200-1-10 | 2055 (25) | 606 (5) | 446 (5) | 2264 (27) |
| 112A-3200-1-58 | 1931 (29) | 399 (4) | 304 (5) | 1693 (25) |
| 112A-3200-1-04 | 1797 (38) | 572 (7) | 260 (6) | 1167 (25) |
| Control* | 378 (9) | 0 (0) | 398 (10) | 1119 (27) |

*Average of 3 injections

To determine if the LIS1::crtB::nos (pBHX112) construct was sexually inheritable, lines 112A-3200-1-53 and 112A-3200-1-45 noted above were crossed as female parents with either ‘Carpet Butter Cream’ petunia, (commercially available from PanAmerican Seed Company, 622 Town Road, West Chicago, Ill.) or a PanAmerican Seed proprietary breeding line 6923-1. Petal and leaf tissues from two progeny 7685 and 7688 were analyzed for carotenoid content following the HPLC procedure described above. Controls were non-transgenic plants from the same cross. For this analysis, leaf tissue was saponified during extraction to remove the chlorophyll. Values for carotenoids shown in Table 11 below are indicated using peak area as percent of the total area at 450 nm. Phytoene was identified based on spectral signature, and phytoene area was determined from a max plot. Data is expressed as normalized peak area.

The inheritability of LIS1 promoter activity in the petal tissue is evident in an observed over 17-fold increase in petal tissue β-carotene levels as compared to controls. As noted above, phytoene was observed in the transgenic petal tissue, but was undetected in control petal tissue. Leaf tissue carotenoid content was not substantially different from control tissue. Zeaxanthin and lutein contents are not significantly different from controls. The LIS1 promoter can be concluded to be inheritable in both marigold and petunia, and to direct high levels of transgene expression in the flowers of both species.

TABLE 11

| | Peak Area | | | |
|---|---|---|---|---|
| Line | β-carotene | phytoene | zeaxanthin | lutein |
| 7685^{a} Leaf | 16657 | | 33 | 34083 |
| 7685 Leaf Control | 12808 | | | 28786 |
| 7685 Petal | 5917 | 2342 | 45 | 1776 |
| 7685 Petal Control | 488 | | 152 | 1235 |
| 7688^{b} Leaf | 15016 | | | 35983 |
| 7688 Leaf Control | 12769 | | 33 | 33059 |
| 7688 Petal | 11926 | 1427 | | 3084 |
| 7688 Petal Control | 683 | | | 1420 |

^{a}112A-3200-1-53 x 6923-1
^{b}112A-3200-1-45 x Carpet Butter Cream

### EXAMPLE 7

### Evaluation of GUS Expression in Transgenic Petunia Lines Transformed with a Plasmid Containing the BAMT Promoter and gusA

To evaluate gene expression in flower tissue using the BAMT promoter, ‘Mitchell’ petunia was transformed with a BAMT::gusA::nos construct. BAMT, benzoic acid carboxyl methyl transferase, is the final enzyme in the biosynthesis of methyl benzoate, a volatile ester known to be the most abundant scent compound in snapdragon flowers. Petunia transformants were generated. Once flowering plants were established in the greenhouse, sample flowers and young leaves from seventeen individual plants were stained with an X-gluc solution to detect GUS activity. A subjective rating system, 0 indicating no visible expression up to 4 representing the highest expression, was used.

As shown in Table 12 below, GUS expression was not detected in petal tissue for any of the petunia transformants. In snapdragon, the majority of BAMT gene activity was found in the upper and lower lobes of the corolla (Dudareva et al., The Plant Cell, 12:949-961 (2000)). Thus it cannot be predicted that a promoter taken from a gene expressed in petal tissue can be used to direct petal expression of foreign genes.

TABLE 12
GUS Expression

| Line | Leaf | Petal | Nectary | Stigma | Anthers/pollen |
|---|---|---|---|---|---|
| BAMT::gusA::nos | | | | | |
| BAMT 0201-2-2 | 0 | 0 | 2 | 2 | 0 |
| BAMT 0201-2-6 | 0 | 0 | ?* | 2 | 0 |
| BAMT 0201-2-7 | 0 | 0 | 4 | 2 | 1 |
| BAMT 0201-2-8 | 0 | 0 | 4 | 4 | 2 |
| BAMT 0201-2-9 | 0 | 0 | 0 | 2 | 0 |
| BAMT 0201-2-10 | 0 | 0 | 1 | 2 | 1 |
| BAMT 0201-2-11 | 0 | 0 | ? | 2 | 1 |
| BAMT 0201-2-12 | 0 | 0 | 0 | 3 | 3 |
| BAMT 0201-2-13 | 0 | 0 | 1 | NA** | 2 |
| BAMT 0201-2-14 | 0 | 0 | ? | 2 | 1 |
| BAMT 0201-2-15 | 0 | 0 | 1 | 2 | 2 |
| BAMT 0201-2-16 | 0 | 0 | 1 | 2 | 0 |
| BAMT 0201-2-17 | 0 | 0 | 2 | 3 | 3 |
| BAMT 0201-2-19 | 0 | 0 | 1 | 2 | 1 |
| BAMT 0201-2-20 | 0 | 0 | 1 | 3 | 3 |
| BAMT 0201-2-21 | 0 | 0 | 0 | 2 | 1 |
| BAMT 0201-2-22 | 0 | 0 | 1 | 2 | 0 |

*Possible Weak Expression
**Not Assayed

All references and patents referred to herein are incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made to the composition, operation and arrangement of the method of the present invention described herein without departing from the concept and scope of the invention.

What is claimed is:

1. An isolated polynucleotide encoding a promoter comprising a sequence selected from the group consisting of: SEQ ID NO:2 and a sequence that hybridizes to SEQ ID NO:2 under stringent conditions and fragments and variants thereof.

2. The polynucleotide of claim 1 wherein the stringent conditions are of low stringency.

3. The polynucleotide of claim 1 wherein the stringent conditions are of high stringency.

4. An isolated polynucleotide comprising a sequence capable of initiating transcription in at least one flower of a plant, wherein said sequence is selected from the group consisting of: SEQ ID NO:2 and a sequence that hybridizes to SEQ ID NO:2 under stringent conditions and fragments and variants thereof.

5. The polynucleotide of claim 4 wherein the stringent conditions are of low stringency.

6. The polynucleotide of claim 4 wherein the stringent conditions are of high stringency.

7. An isolated promoter capable of directing transcription in a flower of plant, wherein said promoter comprises a polynucleotide that has a sequence selected from the group consisting of: SEQ ID NO:2 and a sequence that hybridizes to SEQ ID NO:2 under stringent conditions and fragments and variants thereof.

8. The promoter of claim 7 wherein the stringent conditions are of low stringency.

9. The promoter of claim 7 wherein the stringent conditions are of high stringency.

10. An expression cassette comprising a promoter of claim 7 and a polynucleotide sequence operably linked to said promoter, wherein said promoter is capable of initiating transcription and expression of said polynucleotide sequence in a flower of a plant transformed with said expression cassette.

11. The expression cassette of claim 10 wherein the polynucleotide sequence is inserted into the expression cassette in the sense orientation.

12. The expression cassette of claim 10 wherein the polynucleotide sequence is inserted into the expression cassette in the antisense orientation.

13. An expression vector comprising an expression cassette of claim 10.

14. A plant or plant parts, stably transformed with an expression cassette of claim 10.

15. The plant parts of claim 14 wherein the plant parts are selected from the group consisting of cells, protoplasts, cell tissue cultures, callus, cell clumps, embryos, pollen, ovules, petals, styles, stamens, leaves, roots, root tips and anthers.

16. The plant of claim 14 wherein the plant is a monocotyledonous plant.

17. The plant of claim 16 wherein said monocotyledonous plant is selected from the group consisting of: Amaryllidaceae, Graminae, and Poaceae.

18. The plant of claim 14 wherein the plant is a dicotyledonous plant.

19. The plant of claim 18 wherein said dicotyledonous plant is selected from the group consisting of: Apocynaceae, Asteraceae, Compositae, Balsaminaceae, Begoniaceae, Caryophyllaceae, Chenopodiaceae, Cucurbitaceae, Cruciferae, Gentinaceae, Geraniaceae, Euphorbiaceae, Labiatae, Leguminosae, Liliaceae, Lobeliaceae, Malvaceae, Plumbaginaceae, Polemoniaceae, Primulaceae, Ranunculaceae, Rosaceae, Rubiaceae, Scrophulariaceae, Solanaceae, Umbelliferae, Verbenaceae, and Violaceae.

20. Seed of the plant of claim 14 comprising within their genome said expression cassette.

21. An expression cassette comprising a chimeric promoter and a polynucleotide sequence, wherein said polynucleotide sequence is operably linked to said chimeric promoter, and further wherein said chimeric promoter comprises (a) a first polynucleotide having promoter preferred activity in a flower of a plant, wherein said polynucleotide has a sequence selected from the group consisting of: SEQ ID NO:2 and a sequence that hybridizes to SEQ ID NO:2 under stringent conditions and fragments and variants thereof; and (b) at least a second polynucleotide sequence, wherein said polynucleotide is capable of initiating transcription of a polynucleotide sequence in a plant.

22. The expression cassette of claim 21 wherein the chimeric promoter comprises at least a second polynucleotide sequence that has promoter-preferred activity in a flower of a plant.

23. The expression cassette of claim 21 wherein the chimeric promoter comprises a second polynucleotide sequence that has promoter-preferred activity in a flower of a plant.

24. The expression cassette of claim 21, wherein the chimeric promoter comprises (a) a first polynucleotide having promoter preferred activity in a flower of a plant, wherein said polynucleotide has a sequence selected from the group consisting of: SEQ ID NO:2 and a sequence that hybridizes to SEQ ID NO:2 under stringent conditions and fragments and variants thereof; and (b) a second polynucleotide having promoter-preferred activity in a flower of a plant, wherein said second polynucleotide has a sequence selected from the group consisting of: SEQ ID NO:2 and a sequence that hybridizes to SEQ ID NO:2 under stringent conditions and fragments and variants thereof.

25. The expression cassette of claim 21 wherein the polynucleotide sequence operably linked to the chimeric promoter is inserted into the expression cassette in the sense orientation.

26. The expression cassette of claim 21 wherein the polynucleotide sequence operably linked to the chimeric promoter is inserted into the expression cassette in the antisense orientation.

27. An expression vector comprising an expression cassette of claim 21.

28. A plant, or plant parts, stably transformed with an expression cassette of claim 21.

29. The plant parts of claim 28 wherein the plant parts are selected from the group consisting of cells, protoplasts, cell tissue cultures, callus, cell clumps, embryos, pollen, ovules, petals, styles, stamens, leaves, roots, root tips and anthers.

30. The plant of claim 28 wherein the plant is a monocotyledonous plant.

31. The plant of claim 30 wherein said monocotyledonous plant is selected from the group consisting of: . Amaryllidaceae, Graminae, and Poaceae.

32. The plant of claim 28 wherein the plant is a dicotyledonous plant.

33. The plant of claim 32 wherein said dicotyledonous plant is selected from the group consisting of: Apocynaceae, Asteraceae, Compositae, Balsaminaceae, Begoniaceae, Caryophyllaceae, Chenopodiaceae, Cucurbitaceae, Cruciferae, Gentinaceae, Geraniaceae, Euphorbiaceae, Labiatae, Leguminosae, Liliaceae, Lobeliaceae, Malvaceae, Plumbaginaceae, Polemoniaceae, Primulaceae, Ranunculaceae, Rosaceae, Rubiaceae, Scrophulariaceae, Solanaceae, Umbelliferae, Verbenaceae, and Violaceae.

34. Seed of the plant of claim 28 comprising within their genome said expression cassette.

35. A transgenic plant cell stably transformed with a DNA molecule comprising a promoter capable of initiating transcription in a flower of a plant, wherein said promoter comprises a polynucleotide having a sequence selected from the group consisting of SEQ ID NO:2 and a sequence that hybridizes to SEQ ID NO:2 under stringent conditions and fragments and variants thereof.

36. The transgenic plant cell of claim 35 wherein DNA molecule further comprises a selected coding region operable linked to the promoter.

37. The transgenic plant cell of claim 36 wherein the selected coding region is in the sense orientation.

38. The transgenic plant cell of claim 36 wherein the selected coding region is in the antisense orientation.

39. The transgenic plant cell of claim 35 wherein the stringent conditions are of low stringency.

40. The transgenic plant cell of claim 35 wherein the stringent conditions are of high stringency.

41. The transgenic plant cell of claim 36 wherein said selected coding region encodes an insect resistance protein, a bacterial disease resistance protein, a fungal disease resistance protein, a viral disease resistance protein, an anthocyanin biosynthetic enzyme, a carotenoid biosynthetic enzyme, a floral scent biosynthetic protein, a screenable marker protein or a protein that promotes flower longevity.

42. The transgenic plant cell of claim 36 wherein said selected coding region encodes a screenable marker protein selected from the group consisting of: beta-glucuronidase, beta-lactamase, beta-galactosidase, luciferase, aequorine and green fluorescent protein.

43. The transgenic plant cell of claim 36 wherein said selected coding region encodes an anthocyanin biosynthetic enzyme that produces the compounds: pelargonidin, cyanidin, delphinidin, peonidin, malvidin, and petunidin.

44. The transgenic plant cell of claim 36 wherein said selected coding region encodes a carotenoid biosynthetic enzyme that produces the compounds: phytoene, phytofluene, ζ-carotene, neurosporene, lycopene, γ-carotene, β-carotene, α-cryptoxanthin, β-cryptoxanthin, canthaxanthin, capsanthin, capsorubin, zeaxanthin, violaxanthin, neoxanthin, antheraxanthin, lutein and astaxanthin.

45. A method of expressing a selected protein in a flower of a transgenic plant, the method comprising the steps of:
(a) obtaining an expression vector comprising a selected coding region operably linked to a promoter capable of initiating transcription in a flower of a plant, wherein said promoter comprises a polynucleotide having a sequence selected from the group consisting of SEQ ID NO:2 and a sequence that hybridizes to SEQ ID NO:2 under stringent conditions and fragments and variants thereof;
(b) transforming a recipient plant cell with said vector; and
(c) regenerating a transgenic plant expressing said selected protein from said recipient plant cell.

46. The method of claim 45 wherein the selected coding region is in the sense orientation.

47. The method of claim 45 wherein the selected coding region is in the antisense orientation.

48. The method of claim 45 wherein the stringent conditions are of low stringency.

49. The method of claim 45 wherein the stringent conditions are of high stringency.

50. The method of claim 45 wherein said step of transforming comprises a method selected from the group consisting of: microprojectile bombardment, polyethylene glycol-mediated transformation of protoplasts, electroporation and Agrobacterium-mediated transformation.

51. The method of claim 50 wherein said step of transforming comprises microprojectile bombardment.

52. The method of claim 50 wherein said step of transforming comprises polyethylene glycol-mediated transformation of protoplasts.

53. The method of claim 50 wherein said step of transforming comprises electroporation.

54. The method of claim 50 wherein said step of transforming comprises Agrobacterium-mediated transformation.

55. The method of claim 45 wherein said recipient plant cell is from a monocotyledonous plant.

56. The method of claim 55 wherein the monocotyledonous plant is selected from the group consisting of: Amaryllidaceae, Graminae, and Poaceae.

57. The method of claim 45 wherein said recipient plant cell is from a dicotyledonous plant.

58. The method of claim 57 wherein the dicotyledonous plant is selected from the group consisting of: Apocynaceae, Asteraceae, Compositae, Balsaminaceae, Begoniaceae, Caryophyllaceae, Chenopodiaceae, Cucurbitaceae, Cruciferae, Gentinaceae, Geraniaceae, Euphorbiaceae, Labiatae, Leguminosae, Liliaceae, Lobeliaceae, Malvaceae, Plumbaginaceae, Polemoniaceae, Primulaceae, Ranunculaceae, Rosaceae, Rubiaceae, Scrophulariaceae, Solanaceae, Umbelliferae, Verbenaceae, and Violaceae.
